(19) United States
(12) Patent Application Publication
(10) Pub. No.: US 2025/0092044 A1
(43) Pub. Date: Mar. 20, 2025

(54) Kinase Inhibitors

(71) Applicant: Incyte Corporation, Wilmington, DE (US)

(72) Inventors: Zhitong Zheng, Glen Mills, PA (US); Yanran Ai, West Chester, PA (US); David M. Burns, Plymouth Meeting, PA (US); Kai Liu, Chadds Ford, PA (US); Cooper Ashley Taylor, Wilmington, DE (US); Eddy W. Yue, Landenberg, PA (US)

(21) Appl. No.: 18/827,269

(22) Filed: Sep. 6, 2024

Related U.S. Application Data
(60) Provisional application No. 63/537,341, filed on Sep. 8, 2023.

(51) Int. Cl. C07D 471/14; A61K 31/437; A61K 31/4545; A61K 31/5377; A61K 31/5386; C07D 519/00

(52) U.S. Cl. CPC C07D 471/14; A61K 31/437; A61K 31/4545; A61K 31/5377; A61K 31/5386; C07D 519/00

(57) ABSTRACT

The present application provides heterocyclic compounds that modulate the activity of JAK2, which are useful in the treatment of various diseases, including cancer.

### TECHNICAL FIELD

The present invention provides tricyclic urea compounds that modulate the activity of the JAK2 and are useful in the treatment of diseases related to JAK2, including cancer.

### BACKGROUND

Janus kinase (JAK) 2 plays pivotal roles in signaling by several cytokine receptors. The mutant JAK2 V617F is the most common molecular event associated with myeloproliferative neoplasms. Selective targeting of the JAK2 V617F mutant may be useful for treating various pathologies, while sparing essential JAK2 functions. This application is directed to this need and others.

### SUMMARY

The present invention relates to, inter alia, compounds of Formula I:

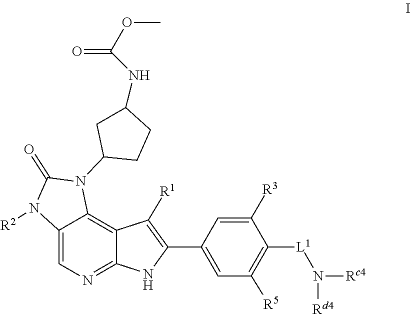

or pharmaceutically acceptable salts thereof, wherein constituent members are defined herein.

The present invention further provides pharmaceutical compositions comprising a compound of Formula I, or a pharmaceutically acceptable salt thereof, and a pharmaceutically acceptable carrier.

The present invention further provides a compound of Formula I, or a pharmaceutically acceptable salt thereof, for use in any of the methods described herein.

The present invention further provides use of a compound of Formula I, or a pharmaceutically acceptable salt thereof, for the preparation of a medicament for use in any of the methods described herein.

### DETAILED DESCRIPTION

The present application provides a compound of Formula I:

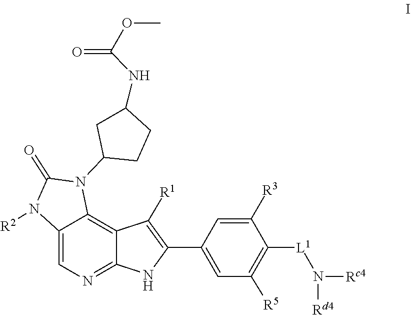

or a pharmaceutically acceptable salt thereof, wherein:
   L¹ is C₁₋₃ alkylene or C(O);
   R¹ is selected from phenyl, dihydrobenzofuranyl, dihydroisobenzofuranyl, and indazolyl, each of which is optionally substituted by 1, 2, or 3 substituents independently selected from halo, C₁₋₆ alkyl, C₁₋₆ haloalkyl, and C₁₋₃ alkoxy-C₁₋₃ alkoxy-;
R² is C₁₋₆ alkyl;
R³ is selected from H, halo, and C₁₋₆ alkoxy;
R^{c4} and R^{d4} are each independently selected from H, C₁₋₆ alkyl, C₁₋₆ haloalkyl, and C₃₋₁₀ cycloalkyl, wherein the C₁₋₆ alkyl and C₃₋₁₀ cycloalkyl are each optionally substituted with 1, 2, 3, or 4 substituents independently selected from halo, C₁₋₆ alkyl, C₁₋₆ haloalkyl, C₁₋₆ alkoxy, and phenylamino;
or R^{c4} and R^{d4}, together with the N atom to which they are attached, form a 4-10 membered heterocycloalkyl group, which is optionally substituted with 1, 2, 3, or 4 substituents independently selected from halo and C₁₋₆ alkoxy; and
R⁵ is selected from H, halo, and C₁₋₆ alkoxy.

In some embodiments, L¹ is C₁₋₃ alkylene.

In some embodiments, L¹ is —CH₂— or C(O).

In some embodiments, L¹ is —CH₂—.

In some embodiments, L¹ is C(O).

In some embodiments, R¹ is selected from phenyl, dihydrobenzofuranyl, dihydroisobenzofuranyl, and indazolyl, each of which is optionally substituted by 1, 2, or 3 substituents independently selected from halo, C₁₋₆ alkyl, and C₁₋₃ alkoxy-C₁₋₃ alkoxy-.

In some embodiments, R¹ is selected from phenyl, dihydrobenzofuranyl, dihydroisobenzofuranyl, and indazolyl, each of which is optionally substituted by 1, 2, or 3 substituents independently selected from halo, C₁₋₃ alkyl, and C₁₋₃ alkoxy-C₁₋₃ alkoxy-.

In some embodiments, R¹ is selected from phenyl, dihydrobenzofuranyl, dihydroisobenzofuranyl, and indazolyl, each of which is optionally substituted by 1 or 2 substituents independently selected from halo, C₁₋₆ alkyl, and C₁₋₃ alkoxy-C₁₋₃ alkoxy-.

In some embodiments, R¹ is selected from phenyl, dihydrobenzofuranyl, dihydroisobenzofuranyl, and indazolyl, each of which is optionally substituted by 1 or 2 substituents independently selected from halo, C₁₋₃ alkyl, and C₁₋₃ alkoxy-C₁₋₃ alkoxy-.

In some embodiments, R¹ is selected from phenyl, dihydrobenzofuranyl, dihydroisobenzofuranyl, and indazolyl, each of which is optionally substituted by 1 or 2 substituents independently selected from fluoro, methyl, isopropyl, and methoxyethoxy.

In some embodiments, R¹ is selected from fluorophenyl, (methoxyethoxy)phenyl, dimethyldihydrobenzofuranyl, dimethyldihydroisobenzofuranyl, and isopropylindazolyl.

In some embodiments, R¹ is fluorophenyl.

In some embodiments, R¹ is (methoxyethoxy)phenyl.

In some embodiments, R¹ is dimethyldihydrobenzofuranyl.

In some embodiments, R¹ is dimethyldihydroisobenzofuranyl.

In some embodiments, R¹ is isopropylindazolyl.

In some embodiments, R¹ is selected from

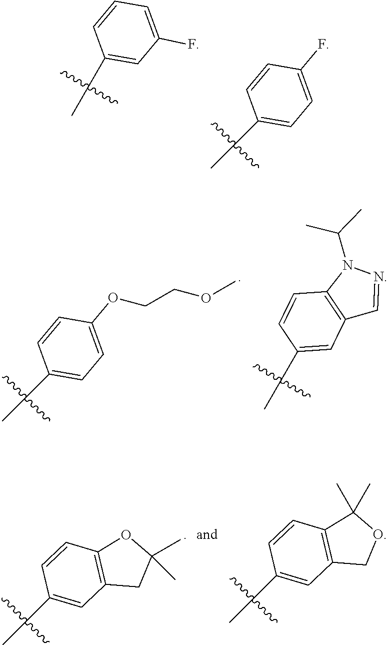 [SMILES: *c1cccc(F)c1 ; *c1ccc(F)cc1 ; *c1ccc(OCCOC)cc1 ; *c1ccc2c(cnn2C(C)C)c1 ; *c1ccc2c(c1)CC(C)(C)O2 ; *c1ccc2c(c1)COC2(C)C]

In some embodiments, R¹ is

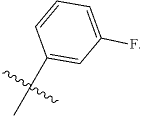 [SMILES: *c1cccc(F)c1]

In some embodiments, R¹ is

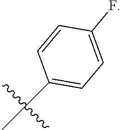 [SMILES: *c1ccc(F)cc1]

In some embodiments, R¹ is

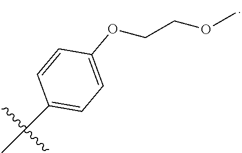 [SMILES: *c1ccc(OCCOC)cc1]

In some embodiments, R¹ is

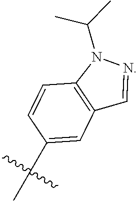 [SMILES: *c1ccc2c(cnn2C(C)C)c1]

In some embodiments, R¹ is

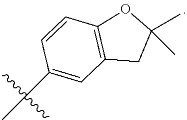 [SMILES: *c1ccc2c(c1)CC(C)(C)O2]

In some embodiments, R¹ is

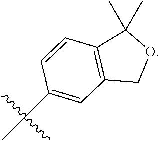 [SMILES: *c1ccc2c(c1)COC2(C)C]

In some embodiments, R² is C₁₋₃ alkyl.

In some embodiments, R² is methyl or trideuteromethyl (i.e., —CD₃).

In some embodiments, R² is trideuteromethyl.

In some embodiments, R² is methyl.

In some embodiments, R³ is selected from H, halo, and C₁₋₃ alkoxy.

In some embodiments, R³ is selected from H, fluoro, and methoxy.

In some embodiments, R³ is H.

In some embodiments, R³ is fluoro.

In some embodiments, R³ is methoxy.

In some embodiments, R^{c4} and R^{d4} are each independently selected from H, C₁₋₆ alkyl, C₁₋₆ haloalkyl, and C₃₋₁₀ cycloalkyl, wherein the C₁₋₆ alkyl and C₃₋₁₀ cycloalkyl are each optionally substituted with 1, 2, 3, or 4 substituents independently selected from halo, C₁₋₆ alkyl, C₁₋₆ haloalkyl, C₁₋₆ alkoxy, and phenylamino.

In some embodiments, R^{c4} and R^{d4} are each independently selected from H, C₁₋₆ alkyl, C₁₋₆ haloalkyl, and C₃₋₇ cycloalkyl, wherein the C₁₋₆ alkyl and C₃₋₇ cycloalkyl are each optionally substituted with 1, 2, 3, or 4 substituents independently selected from halo, C₁₋₃ alkyl, C₁₋₃ haloalkyl, C₁₋₃ alkoxy, and phenylamino.

In some embodiments, R^{c4} and R^{d4} are each independently selected from H, C₁₋₃ alkyl, C₁₋₃ haloalkyl, and C₃₋₇ cycloalkyl, wherein the C₁₋₃ alkyl and C₃₋₇ cycloalkyl are each optionally substituted with 1, 2, 3, or 4 substituents independently selected from halo, C₁₋₃ alkyl, C₁₋₃ haloalkyl, C₁₋₃ alkoxy, and phenylamino.

In some embodiments, R^{c4} and R^{d4} are each independently selected from H, methyl, trideuteromethyl, ethyl, propyl, isopropyl, trifluoroethyl, and cyclopropyl, wherein the methyl, ethyl, propyl, isopropyl, and cyclopropyl are each optionally substituted with 1, 2, 3, or 4 substituents independently selected from fluoro, trifluoromethyl, methoxy, and phenylamino.

In some embodiments, R^{c4} and R^{d4} are each independently selected from H, methyl, trideuteromethyl, ethyl, propyl, isopropyl, trifluoroethyl, and cyclopropyl, wherein the methyl, ethyl, propyl, isopropyl, and cyclopropyl are each optionally substituted with 1 or 2 substituents independently selected from fluoro, trifluoromethyl, methoxy, and phenylamino.

In some embodiments, R^{c4} is selected from H, C₁₋₆ alkyl, C₁₋₆ haloalkyl, and C₃₋₁₀ cycloalkyl, wherein the C₁₋₆ alkyl and C₃₋₁₀ cycloalkyl are each optionally substituted with 1, 2, 3, or 4 substituents independently selected from halo, C₁₋₆ alkyl, C₁₋₆ haloalkyl, C₁₋₆ alkoxy, and phenylamino.

In some embodiments, R^{c4} is selected from H, C₁₋₆ alkyl, C₁₋₆ haloalkyl, and C₃₋₇ cycloalkyl, wherein the C₁₋₆ alkyl and C₃₋₇ cycloalkyl are each optionally substituted with 1, 2, 3, or 4 substituents independently selected from halo, C₁₋₃ alkyl, C₁₋₃ haloalkyl, C₁₋₃ alkoxy, and phenylamino.

In some embodiments, R^{c4} is selected from H, C₁₋₃ alkyl, C₁₋₃ haloalkyl, and C₃₋₇ cycloalkyl, wherein the C₁₋₃ alkyl and C₃₋₇ cycloalkyl are each optionally substituted with 1, 2, 3, or 4 substituents independently selected from halo, C₁₋₃ alkyl, C₁₋₃ haloalkyl, C₁₋₃ alkoxy, and phenylamino.

In some embodiments, R^{c4} is selected from methyl, trideuteromethyl, ethyl, propyl, isopropyl, trifluoroethyl, and cyclopropyl, wherein the methyl, ethyl, propyl, isopropyl, and cyclopropyl are each optionally substituted with 1, 2, 3, or 4 substituents independently selected from fluoro, trifluoromethyl, methoxy, and phenylamino.

In some embodiments, R^{c4} is selected from methyl, trideuteromethyl, ethyl, propyl, isopropyl, trifluoroethyl, and cyclopropyl, wherein the methyl, ethyl, propyl, isopropyl, and cyclopropyl are each optionally substituted with 1 or 2 substituents independently selected from fluoro, trifluoromethyl, methoxy, and phenylamino.

In some embodiments, R^{d4} is selected from H and C₁₋₆ alkyl.

In some embodiments, R⁴⁴ is selected from H and C₁₋₃ alkyl.

In some embodiments, R^{d4} is selected from H, methyl, and trideuteromethyl.

In some embodiments:
R^{c4} is selected from methyl, trideuteromethyl, ethyl, propyl, isopropyl, trifluoroethyl, and cyclopropyl, wherein the methyl, ethyl, propyl, isopropyl, and cyclopropyl are each optionally substituted with 1, 2, 3, or 4 substituents independently selected from fluoro, trifluoromethyl, methoxy, and phenylamino; and
R^{d4} is selected from H, methyl, and trideuteromethyl.

In some embodiments:
R^{c4} is selected from methyl, trideuteromethyl, ethyl, isopropyl, trifluoroethyl, methoxyethyl, cyclopropyl, phenylaminopropyl, difluorocyclopropyl, and trifluoromethylcyclopropyl; and
R^{d4} is selected from H and C₁₋₆ alkyl.

In some embodiments:
R^{c4} is selected from methyl, trideuteromethyl, ethyl, isopropyl, trifluoroethyl, methoxyethyl, cyclopropyl, phenylaminopropyl, difluorocyclopropyl, and trifluoromethylcyclopropyl; and
R⁴⁴ is selected from H and C₁₋₃ alkyl.

In some embodiments:
R^{c4} is selected from methyl, ethyl, isopropyl, trifluoroethyl, methoxyethyl, cyclopropyl, phenylaminopropyl, difluorocyclopropyl, and trifluoromethylcyclopropyl; and
R^{d4} is selected from H, methyl, and trideuteromethyl.

In some embodiments, R^{c4} and R^{d4}, together with the N atom to which they are attached, form a 4-10 membered heterocycloalkyl group, which is optionally substituted with 1, 2, 3, or 4 substituents independently selected from halo and C₁₋₆ alkoxy.

In some embodiments, R^{c4} and R^{d4}, together with the N atom to which they are attached, form a monocyclic 4-7 membered heterocycloalkyl group or a 8-10 membered bicyclic heterocycloalkyl group, wherein the monocyclic 4-7 membered heterocycloalkyl group and 8-10 membered bicyclic heterocycloalkyl group are each optionally substituted with 1, 2, 3, or 4 substituents independently selected from halo and C₁₋₆ alkoxy.

In some embodiments, R^{c4} and R^{d4}, together with the N atom to which they are attached, form a monocyclic 4-7 membered heterocycloalkyl group or a 8-10 membered bicyclic heterocycloalkyl group, wherein the monocyclic 4-7 membered heterocycloalkyl group and 8-10 membered bicyclic heterocycloalkyl group are each optionally substituted with 1, 2, 3, or 4 substituents independently selected from halo and C₁₋₃ alkoxy.

In some embodiments, R^{c4} and R^{d4}, together with the N atom to which they are attached, form azetidinyl, piperidinyl, morpholinyl, or 3-oxa-8-azabicyclo[3.2.1]octanyl, wherein the azetidinyl, piperidinyl, morpholinyl, and 3-oxa-8-azabicyclo[3.2.1]octanyl are each optionally substituted with 1, 2, 3, or 4 substituents independently selected from fluoro and methoxy.

In some embodiments, R^{c4} and R^{d4}, together with the N atom to which they are attached, form methoxyazetidinyl, difluoropiperidinyl, morpholinyl, or 3-oxa-8-azabicyclo[3.2.1]octanyl.

In some embodiments, R⁵ is selected from H, halo, and C₁₋₃ alkoxy.

In some embodiments, R⁵ is selected from H, fluoro, and methoxy.

In some embodiments, R⁵ is H.

In some embodiments, R⁵ is fluoro.

In some embodiments, R⁵ is methoxy.

In some embodiments:
R³ is selected from H, halo, and C₁₋₆ alkoxy; and
R⁵ is selected from H and halo.

In some embodiments:
R³ is selected from H, halo, and C₁₋₃ alkoxy; and
R⁵ is selected from H and halo.

In some embodiments:
R³ is selected from H, fluoro, and methoxy; and
R⁵ is selected from H and fluoro.

In some embodiments:
L¹ is —CH₂— or C(O);
R¹ is selected from phenyl, dihydrobenzofuranyl, dihydroisobenzofuranyl, and indazolyl, each of which is optionally substituted by 1 or 2 substituents independently selected from fluoro, methyl, isopropyl, and methoxyethoxy;
R² is C₁₋₃ alkyl;
R^{c4} and R^{d4} are each independently selected from H, C₁₋₆ alkyl, C₁₋₆ haloalkyl, and C₃₋₁₀ cycloalkyl, wherein the C₁₋₆ alkyl and C₃₋₁₀ cycloalkyl are each optionally substituted with 1, 2, 3, or 4 substituents independently selected from halo, C₁₋₆ alkyl, C₁₋₆ haloalkyl, C₁₋₆ alkoxy, and phenylamino; or
R^{c4} and R^{d4}, together with the N atom to which they are attached, form a 4-10 membered heterocycloalkyl group, which is optionally substituted with 1, 2, 3, or 4 substituents independently selected from halo and C₁₋₆ alkoxy; and
R⁵ is selected from H, halo, and C₁₋₃ alkoxy.

In some embodiments:
L¹ is —CH₂— or C(O);
R¹ is selected from phenyl, dihydrobenzofuranyl, dihydroisobenzofuranyl, and indazolyl, each of which is optionally substituted by 1 or 2 substituents independently selected from fluoro, methyl, isopropyl, and methoxyethoxy;
R² is C₁₋₃ alkyl;
R^{c4} and R^{d4} are each independently selected from H, C₁₋₃ alkyl, C₁₋₃ haloalkyl, and C₃₋₇ cycloalkyl, wherein the C₁₋₃ alkyl and C₃₋₇ cycloalkyl are each optionally substituted with 1, 2, 3, or 4 substituents independently selected from halo, C₁₋₃ alkyl, C₁₋₃ haloalkyl, C₁₋₃ alkoxy, and phenylamino; or
R^{c4} and R^{d4}, together with the N atom to which they are attached, form a monocyclic 4-7 membered heterocycloalkyl group or a 8-10 membered bicyclic heterocycloalkyl group, wherein the monocyclic 4-7 membered heterocycloalkyl group and 8-10 membered bicyclic heterocycloalkyl group are each optionally substituted with 1, 2, 3, or 4 substituents independently selected from halo and C₁₋₆ alkoxy; and
R⁵ is selected from H, halo, and C₁₋₃ alkoxy.

In some embodiments:
L¹ is —CH₂— or C(O);
R¹ is selected from fluorophenyl, (methoxyethoxy)phenyl, dimethyldihydrobenzofuranyl, dimethyldihydroisobenzofuranyl, and isopropylindazolyl;
R² is methyl or trideuteromethyl;
R³ is selected from H, fluoro, and methoxy;
R^{c4} and R^{d4} are each independently selected from H, methyl, trideuteromethyl, ethyl, propyl, isopropyl, trifluoroethyl, and cyclopropyl, wherein the methyl, ethyl, propyl, isopropyl, and cyclopropyl are each optionally substituted with 1, 2, 3, or 4 substituents independently selected from fluoro, trifluoromethyl, methoxy, and phenylamino; or
R^{c4} and R^{d4}, together with the N atom to which they are attached, form azetidinyl, piperidinyl, morpholinyl, or 3-oxa-8-azabicyclo[3.2.1]octanyl, wherein the azetidinyl, piperidinyl, morpholinyl, and 3-oxa-8-azabicyclo[3.2.1]octanyl are each optionally substituted with 1, 2, 3, or 4 substituents independently selected from fluoro and methoxy; and
R⁵ is selected from H, fluoro, and methoxy.

In some embodiments, the compound of Formula I is a compound of Formula Ia:

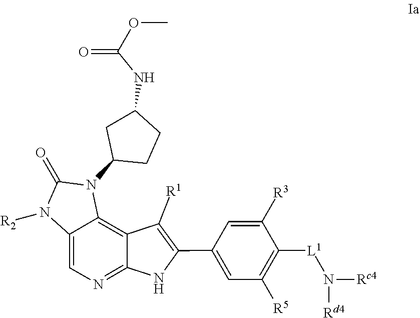

or a pharmaceutically acceptable salt thereof.

In some embodiments, the compound of Formula I is a compound of Formula Ib:

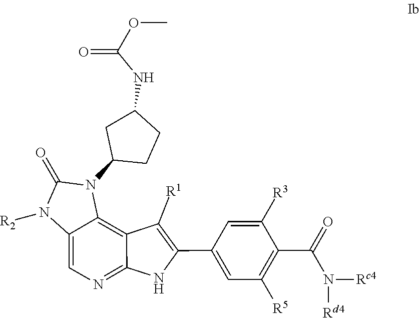

or a pharmaceutically acceptable salt thereof.

In some embodiments, the compound of Formula I is a compound of Formula Ic:

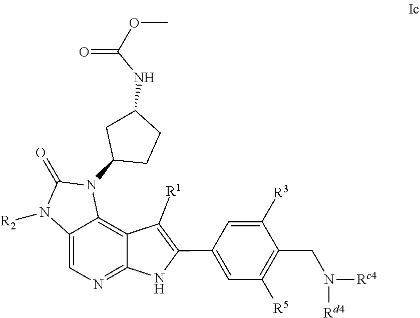

or a pharmaceutically acceptable salt thereof.

In some embodiments, the compound of Formula I is a compound of Formula II:

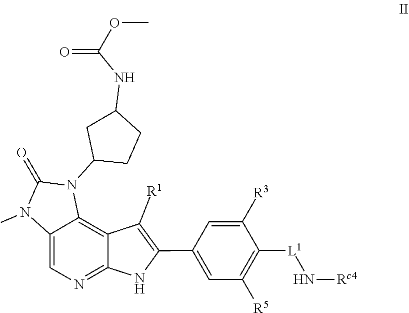

or a pharmaceutically acceptable salt thereof.

In some embodiments, the compound of Formula I is a compound of Formula IIa:

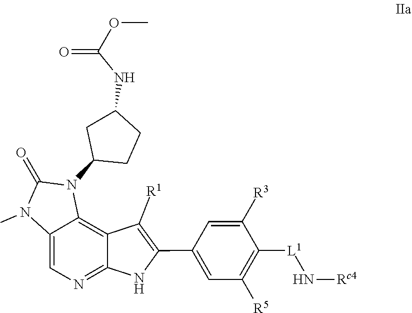

or a pharmaceutically acceptable salt thereof.

In some embodiments, the compound of Formula I is a compound of Formula IIb:

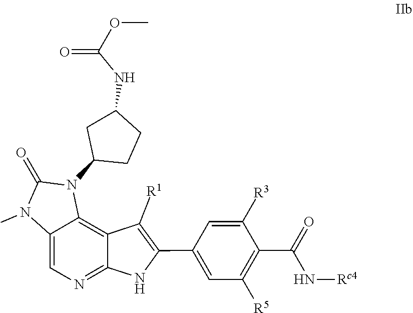

or a pharmaceutically acceptable salt thereof.

In some embodiments, the compound of Formula I is a compound of Formula IIc:

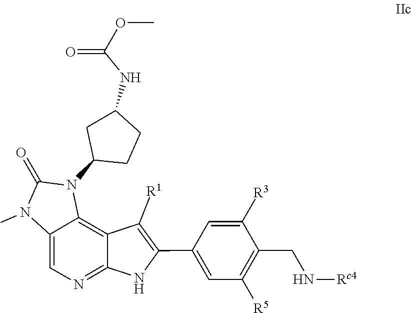

or a pharmaceutically acceptable salt thereof.

In some embodiments, the compound of Formula I is a compound of Formula III:

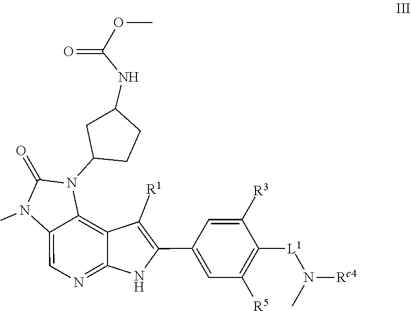

or a pharmaceutically acceptable salt thereof.

In some embodiments, the compound of Formula I is a compound of Formula IIIa:

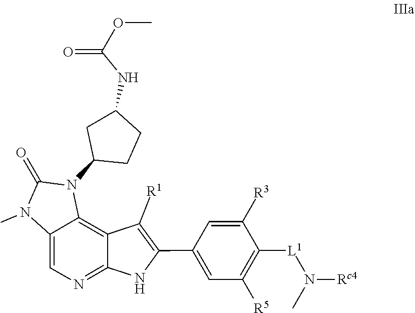

or a pharmaceutically acceptable salt thereof.

In some embodiments, the compound of Formula I is a compound of Formula IIIb:

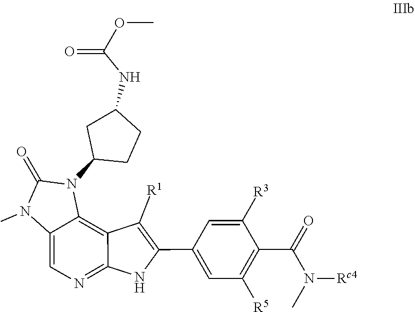

or a pharmaceutically acceptable salt thereof.

In some embodiments, the compound of Formula I is a compound of Formula IIIc:

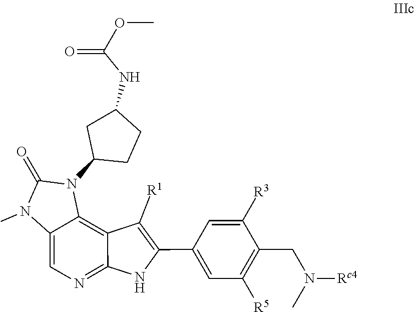

or a pharmaceutically acceptable salt thereof.

In some embodiments, the compound of Formula I is a compound of Formula IV:

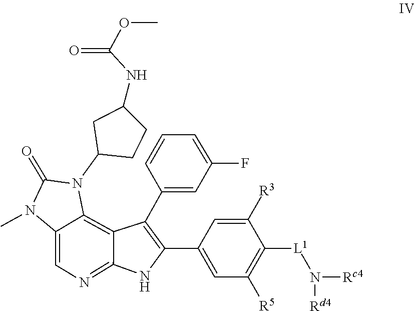

or a pharmaceutically acceptable salt thereof.

In some embodiments, the compound of Formula I is a compound of Formula IVa:

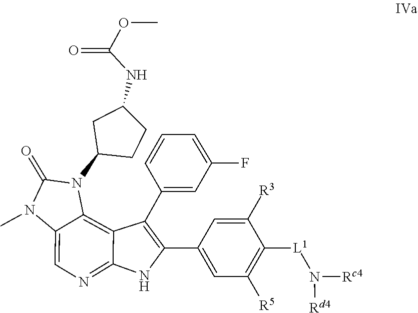

or a pharmaceutically acceptable salt thereof.

In some embodiments, the compound of Formula I is a compound of Formula IVb:

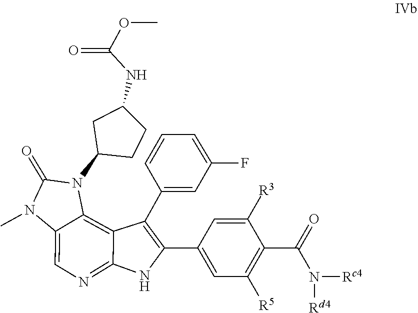

or a pharmaceutically acceptable salt thereof.
pharmaceutically acceptable salt thereof.

In some embodiments, the compound of Formula I is a compound of Formula IVc:

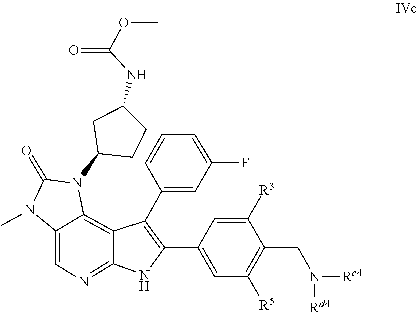

or a pharmaceutically acceptable salt thereof.

In some embodiments, the compound of Formula I is a compound of Formula V:

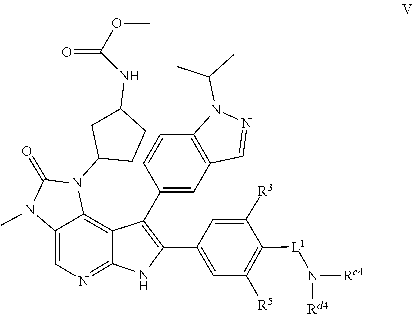

or a pharmaceutically acceptable salt thereof.

In some embodiments, the compound of Formula I is a compound of Formula Va:

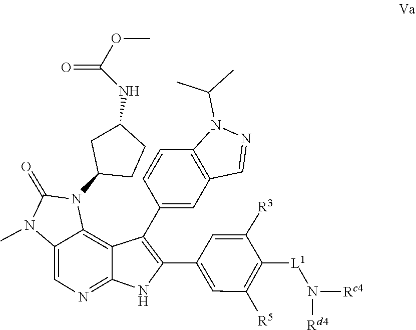

or a pharmaceutically acceptable salt thereof.

In some embodiments, the compound of Formula I is a compound of Formula Vb:

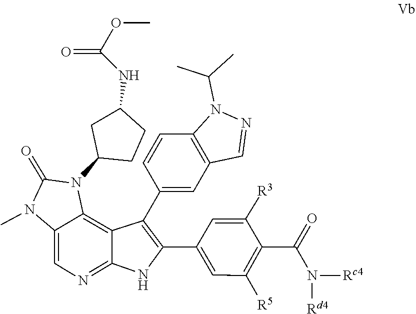

or a pharmaceutically acceptable salt thereof.

In some embodiments, the compound of Formula I is a compound of Formula Vc:

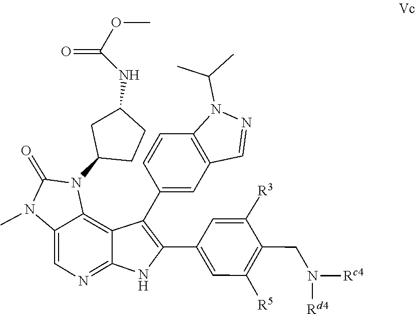

or a pharmaceutically acceptable salt thereof.

In some embodiments, the compound of Formula I is a compound of Formula VI:

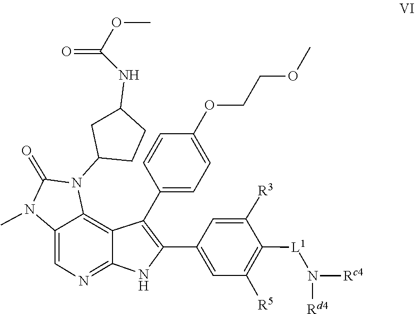

or a pharmaceutically acceptable salt thereof.

In some embodiments, the compound of Formula I is a compound of Formula VIa:

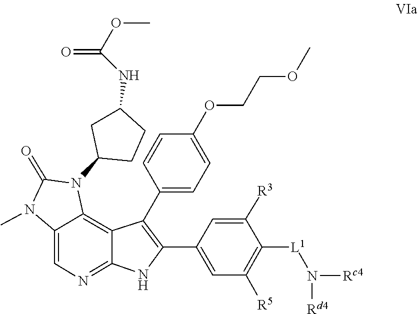

or a pharmaceutically acceptable salt thereof.

In some embodiments, the compound of Formula I is a compound of Formula VIb:

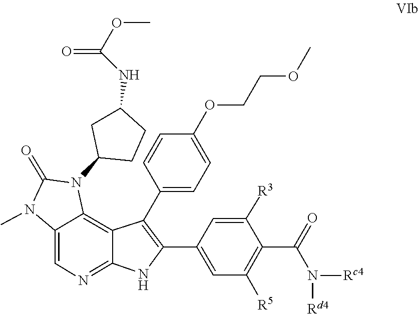

or a pharmaceutically acceptable salt thereof.

In some embodiments, the compound of Formula I is a compound of Formula VIc:

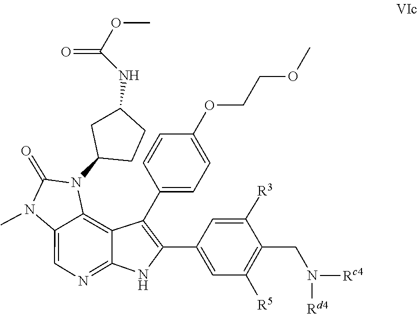

or a pharmaceutically acceptable salt thereof.

In some embodiments, the compound of Formula I is a compound of Formula VII:

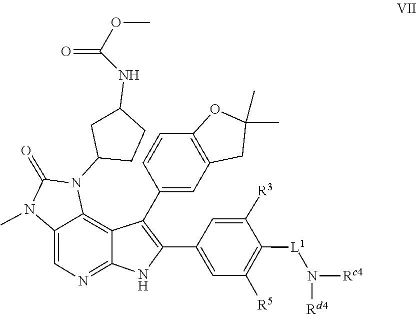

or a pharmaceutically acceptable salt thereof.

In some embodiments, the compound of Formula I is a compound of Formula VIIa:

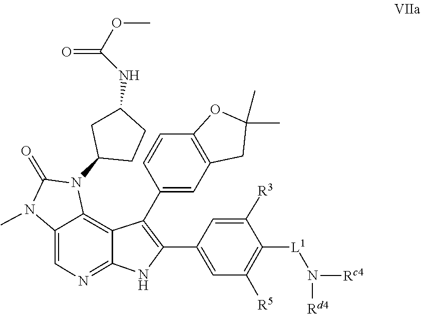

or a pharmaceutically acceptable salt thereof.

In some embodiments, the compound of Formula I is a compound of Formula VIIb:

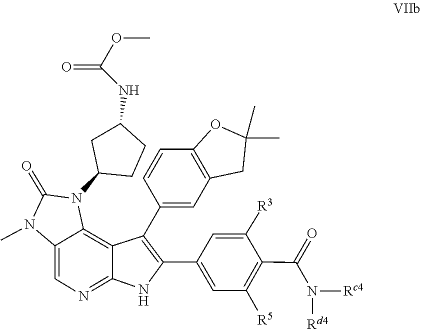

or a pharmaceutically acceptable salt thereof.

In some embodiments, the compound of Formula I is a compound of Formula VIIc:

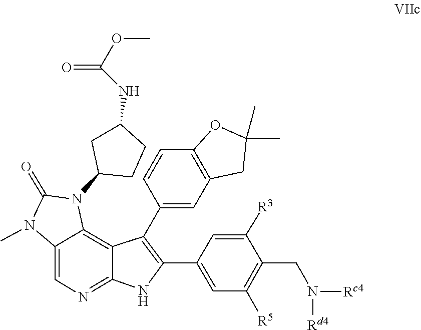

or a pharmaceutically acceptable salt thereof.

In some embodiments, the compound of Formula I is a compound of Formula VIII:

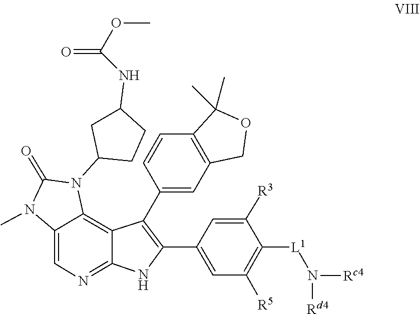

or a pharmaceutically acceptable salt thereof.

In some embodiments, the compound of Formula I is a compound of Formula VIIIa:

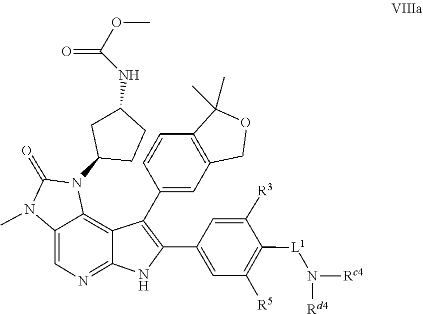

or a pharmaceutically acceptable salt thereof.

In some embodiments, the compound of Formula I is a compound of Formula VIIIb:

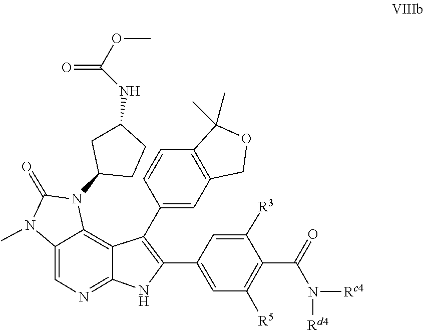

or a pharmaceutically acceptable salt thereof.

In some embodiments, the compound of Formula I is a compound of Formula VIIIc:

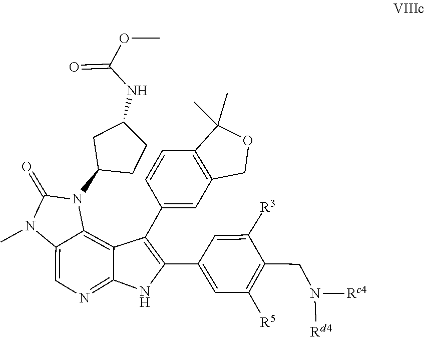

or a pharmaceutically acceptable salt thereof.

In some embodiments, the compound provided herein is selected from:
methyl ((1R,3R)-3-(7-(3-fluoro-4-(methylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(isopropylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3,5-difluoro-4-(methylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(cyclopropylcarbamoyl)-3,5-difluorophenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(3-oxa-8-azabicyclo[3.2.1]octane-8-carbonyl)-3-methoxyphenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(ethylcarbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((3-(phenylamino)propyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(cyclopropylcarbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-((2,2-difluorocyclopropyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2-(trifluoromethyl)cyclopropyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2,2,2-trifluoroethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(methylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((1-(trifluoromethyl)cyclopropyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2-methoxyethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2-methoxyethyl)(methyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(morpholine-4-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(3-methoxyazetidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(ethylcarbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(8-(1-isopropyl-1H-indazol-5-yl)-7-(4-((2-methoxyethyl)carbamoyl)phenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(8-(1-isopropyl-1H-indazol-5-yl)-3-methyl-7-(4-(morpholine-4-carbonyl)phenyl)-2-oxo-3,6-dihydroimidazo[4,5-d]pyrrolo[2,3-b]pyridin-1(2H)-yl)cyclopentyl)carbamate;
methyl ((1R,3R)-3-(8-(1-isopropyl-1H-indazol-5-yl)-3-methyl-7-(4-(methylcarbamoyl)phenyl)-2-oxo-3,6-dihydroimidazo[4,5-d]pyrrolo[2,3-b]pyridin-1(2H)-yl)cyclopentyl)carbamate;
methyl ((1R,3R)-3-(7-(4-(dimethylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(ethylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(4,4-difluoropiperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-((bis(methyl-d3)amino)methyl)phenyl)-8-(2,2-dimethyl-2,3-dihydrobenzofuran-5-yl)-3-(methyl-d3)-2-oxo-3,6-dihydroimidazo[4,5-d]pyrrolo[2,3-b]pyridin-1(2H)-yl)cyclopentyl)carbamate;
methyl ((1R,3R)-3-(7-(4-((dimethylamino)methyl)phenyl)-8-(1-isopropyl-1H-indazol-5-yl)-3-(methyl-d3)-2-oxo-3,6-dihydroimidazo[4,5-d]pyrrolo[2,3-b]pyridin-1(2H)-yl)cyclopentyl)carbamate;
methyl ((1R,3R)-3-(7-(4-((bis(methyl-d3)amino)methyl)phenyl)-8-(4-(2-methoxyethoxy)phenyl)-3-(methyl-d3)-2-oxo-3,6-dihydroimidazo[4,5-d]pyrrolo[2,3-b]pyridin-1(2H)-yl)cyclopentyl)carbamate;
methyl ((1R,3R)-3-(7-(4-((dimethylamino)methyl)phenyl)-8-(4-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-((bis(methyl-d3)amino)methyl)phenyl)-8-(1,1-dimethyl-1,3-dihydroisobenzofuran-5-yl)-3-(methyl-d3)-2-oxo-3,6-dihydroimidazo[4,5-d]pyrrolo[2,3-b]pyridin-1(2H)-yl)cyclopentyl)carbamate; and
methyl ((1R,3R)-3-(8-(1,1-dimethyl-1,3-dihydroisobenzofuran-5-yl)-7-(4-(dimethylcarbamoyl)phenyl)-3-(methyl-d3)-2-oxo-3,6-dihydroimidazo[4,5-d]pyrrolo[2,3-b]pyridin-1(2H)-yl)cyclopentyl)carbamate;
   or a pharmaceutically acceptable salt thereof.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, divalent linking substituents are described. It is specifically intended that each divalent linking substituent include both the forward and backward forms of the linking substituent. For example, —NR(CR′R″)ₙ— includes both —NR(CR′R″)ₙ— and —(CR′R″)ₙNR—. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

As used herein, the phrase “each ‘variable’ is independently selected from” means substantially the same as wherein “at each occurrence ‘variable’ is selected from.”

Throughout the definitions, the term “Cₙ₋ₘ” indicates a range which includes the endpoints, wherein n and m are integers and indicate the number of carbons. Examples include C₁₋₃, C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (iPr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, the term “Cₒ₋ₚ alkoxy-Cₙ₋ₘ alkoxy-”, employed alone or in combination with other terms, refers to a group of formula (Cₒ₋ₚ alkyl)-O—(Cₙ₋ₘ alkyl)-O—, wherein the Cₒ₋ₚ alkyl has o to m carbons and the Cₙ₋ₘ alkyl has n to m carbons. Example Cₒ₋ₚ alkoxy-Cₙ₋ₘ alkoxy-groups include, but are not limited to, methoxymethoxy (i.e., CH₃OCH₂O—), methoxyethoxy (i.e., CH₃OCH₂CH₂O—), ethoxymethoxy (i.e., CH₃CH₂OCH₂O—), ethoxyethoxy (i.e., CH₃CH₂OCH₂CH₂O—), methoxypropoxy (i.e., CH₃OCH₂CH₂CH₂O—), and the like.

As used herein, the term “amino” refers to a group of formula —NH₂.

As used herein, “halo” refers to F, Cl, Br, or I. In some embodiments, a halo is F, Cl, or Br. In some embodiments, a halo is F or Cl. In some embodiments, a halo is F. In some embodiments, a halo is Cl.

As used herein, the term “Cₙ₋ₘ haloalkyl”, employed alone or in combination with other terms, refers to an alkyl group having from one halogen atom to 2 s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms. Example haloalkyl groups include CF₃, C₂F₅, CHF₂, CH₂F, CCl₃, CHCl₂, C₂Cl₅ and the like.

As used herein, the term “carbonyl”, employed alone or in combination with other terms, refers to a —C(O)— group.

As used herein, “cycloalkyl” refers to non-aromatic cyclic hydrocarbons including cyclized alkyl and alkenyl groups. Cycloalkyl groups can include mono- or polycyclic (e.g., having 2 fused rings) groups, spirocycles, and bridged rings (e.g., a bridged bicycloalkyl group). Ring-forming carbon atoms of a cycloalkyl group can be optionally substituted by oxo or sulfido (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (i.e., C₃₋₁₀). In some embodiments, the cycloalkyl is a C₃₋₁₀ monocyclic or bicyclic cycloalkyl. In some embodiments, the cycloalkyl is a C₃₋₇ monocyclic cycloalkyl. In some embodiments, the cycloalkyl is a C₄₋₇ monocyclic cycloalkyl. In some embodiments, the cycloalkyl is a C₄₋₁₀ spirocycle or bridged cycloalkyl (e.g., a bridged bicycloalkyl group). Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cubane, adamantane, bicyclo[1.1.1]pentyl, bicyclo[2.1.1]hexyl, bicyclo[2.2.1]heptanyl, bicyclo[3.1.1]heptanyl, bicyclo[2.2.2]octanyl, spiro[3.3]heptanyl, azaspiro[2.4]heptanyl, and the like. In some embodiments, cycloalkyl is cyclopropyl, cyclobutyl, cyclopentyl, or cyclohexyl. and the like.

As used herein, “heterocycloalkyl” refers to monocyclic or polycyclic heterocycles having at least one non-aromatic ring (saturated or partially unsaturated ring), wherein one or more of the ring-forming carbon atoms of the heterocycloalkyl is replaced by a heteroatom selected from N, O, S, and B, and wherein the ring-forming carbon atoms and heteroatoms of a heterocycloalkyl group can be optionally substituted by one or more oxo or sulfido (e.g., C(O), S(O), C(S), or S(O)₂, etc.). When a ring-forming carbon atom or heteroatom of a heterocycloalkyl group is optionally substituted by one or more oxo or sulfide, the O or S of said group is in addition to the number of ring-forming atoms specified herein (e.g., a 1-methyl-6-oxo-1,6-dihydropyridazin-3-yl is a 6-membered heterocycloalkyl group, wherein a ring-forming carbon atom is substituted with an oxo group, and wherein the 6-membered heterocycloalkyl group is further substituted with a methyl group). Heterocycloalkyl groups include monocyclic and polycyclic (e.g., having 2 fused rings) systems. Included in heterocycloalkyl are monocyclic and polycyclic 3 to 10, 4 to 10, 5 to 10, 4 to 7, 5 to 7, or 5 to 6 membered heterocycloalkyl groups. Heterocycloalkyl groups can also include spirocycles and bridged rings (e.g., a 5 to 10 membered bridged biheterocycloalkyl ring having one or more of the ring-forming carbon atoms replaced by a heteroatom independently selected from N, O, S, and B). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds.

Also included in the definition of heterocycloalkyl are moieties that have one or more aromatic rings fused (i.e., having a bond in common with) to the non-aromatic heterocyclic ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl group contains 3 to 10 ring-forming atoms, 4 to 10 ring-forming atoms, 3 to 7 ring-forming atoms, or 5 to 6 ring-forming atoms. In some embodiments, the heterocycloalkyl group has 1 to 4 heteroatoms, 1 to 3 heteroatoms, 1 to 2 heteroatoms or 1 heteroatom. In some embodiments, the heterocycloalkyl is a monocyclic 4-6 membered heterocycloalkyl having 1 or 2 heteroatoms independently selected from N, O, S and B and having one or more oxidized ring members. In some embodiments, the heterocycloalkyl is a monocyclic or bicyclic 5-10 membered heterocycloalkyl having 1, 2, 3, or 4 heteroatoms independently selected from N, O, S, and B and having one or more oxidized ring members. In some embodiments, the heterocycloalkyl is a monocyclic or bicyclic 5 to 10 membered heterocycloalkyl having 1, 2, 3, or 4 heteroatoms independently selected from N, O, and S and having one or more oxidized ring members. In some embodiments, the heterocycloalkyl is a monocyclic 5 to 6 membered heterocycloalkyl having 1, 2, 3, or 4 heteroatoms independently selected from N, O, and S and having one or more oxidized ring members.

Example heterocycloalkyl groups include pyrrolidin-2-one (or 2-oxopyrrolidinyl), 1,3-isoxazolidin-2-one, pyranyl, tetrahydropyranyl, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, 1,2,3,4-tetrahydroisoquinoline, benzazapene, azabicyclo[3.1.0]hexanyl, diazabicyclo[3.1.0]hexanyl, oxobicyclo[2.1.1]hexanyl, azabicyclo[2.2.1]heptanyl, diazabicyclo[2.2.1]heptanyl, azabicyclo[3.1.1]heptanyl, diazabicyclo[3.1.1]heptanyl, azabicyclo[3.2.1]octanyl, diazabicyclo[3.2.1]octanyl, oxobicyclo[2.2.2]octanyl, azabicyclo[2.2.2]octanyl, azaadamantanyl, diazaadamantanyl, oxo-adamantanyl, azaspiro[3.3]heptanyl, diazaspiro[3.3]heptanyl, oxo-azaspiro[3.3]heptanyl, azaspiro[3.4]octanyl, diazaspiro[3.4]octanyl, oxo-azaspiro[3.4]octanyl, azaspiro[2.5]octanyl, diazaspiro[2.5]octanyl, azaspiro[4.4]nonanyl, diazaspiro[4.4]nonanyl, oxo-azaspiro[4.4]nonanyl, azaspiro[4.5]decanyl, diazaspiro[4.5]decanyl, diazaspiro[4.4]nonanyl, oxo-diazaspiro[4.4]nonanyl, oxo-dihydropyridazinyl, oxo-2,6-diazaspiro[3.4]octanyl, oxohexahydropyrrolo[1,2-a]pyrazinyl, 5,6-dihydro-4H-pyrrolo[1,2-b]pyrazolyl, 3-oxopiperazinyl, oxo-pyrrolidinyl, oxo-pyridinyl, 2,3-dihydrobenzo[b][1,4]dioxinyl, 3,4-dihydro-2H-benzo[b][1,4]oxazinyl, octahydro-2H-pyrido[1,2-a]pyrazinyl, 1,6-diazaspiro[3.3]heptanyl, 3-azabicyclo[3.1.0]hexanyl, 2-oxa-5-azabicyclo[2.2.1]heptanyl, and 2-oxa-5-azabicyclo[4.1.0]heptanyl, and the like.

As used herein, an “alkyl linking group” is a bivalent straight chain or branched alkyl linking group (“alkylene group”). For example, “Cₒ₋ₚ cycloalkyl-Cₙ₋ₘ alkyl-”, “Cₒ₋ₚ aryl-Cₙ₋ₘ alkyl-”, “phenyl-Cₙ₋ₘ alkyl-”, “heteroaryl-Cₙ₋ₘ alkyl-”, and “heterocycloalkyl-Cₙ₋ₘ alkyl-” contain alkyl linking groups. Examples of “alkyl linking groups” or “alkylene groups” include methylene, ethan-1,1-diyl, ethan-1,2-diyl, propan-1,3-dilyl, propan-1,2-diyl, propan-1,1-diyl and the like.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

As used herein, the term “oxo” refers to an oxygen atom (i.e., ═O) as a divalent substituent, forming a carbonyl group when attached to a carbon (e.g., C═O or C(O)), or attached to a nitrogen or sulfur heteroatom forming a nitroso, sulfinyl, or sulfonyl group.

As used herein, the term “independently selected from” means that each occurrence of a variable or substituent (e.g., each R^{M}), are independently selected at each occurrence from the applicable list.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula I, Formula IIa, etc.) provided herein include stereoisomers of the compounds.

Resolution of racemic mixtures of compounds can be carried out by any of numerous methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes.

Compounds of Formula I can be prepared, for example, as shown in Scheme 1. Optionally protected (e.g., P=phenylsulfonyl) nitrobicyclic heterocycles 1-1 where X¹ is halogen (e.g., Cl, Br, or I) or pseudohalogen (e.g., OTf or OMs) can be converted to dihalide 1-2, where X² is a halogen (e.g., Cl, Br, or I) under standard halogenation conditions (e.g., in the presence of NBS or NIS or NCS). Dihalide 1-2 can be coupled with amines such as 1-3 under standard S_{N}Ar conditions (e.g., in the presence of a base such as Et₃N) to give 1-4. Nitroheterocycle 1-4 can be reduced under standard conditions (e.g., in the presence of Fe and acid) to give 1-5. Diamine 1-5 can be converted to cyclic urea 1-6 under standard conditions (e.g., in the presence of CDI). Cyclic urea 1-6 can be alkylated under standard S_{N}2 conditions (e.g., in the presence of K₂CO₃ and R²—Y¹, where Y¹ is a halogen (e.g., Cl, Br, or I) or pseudohalogen (e.g., OTf or OMs)) to give 1-7. The Boc group in compound 1-7 can be removed under standard conditions (e.g., TFA in CH₂Cl₂) to give amine 1-8. Amine 1-8 can be converted to carbamate 1-9 under standard conditions (e.g., methyl chloroformate). Compound 1-9 can be coupled with R¹-M¹, where M¹ is a boronic acid, boronate ester, potassium trifluoroborate, or an appropriately substituted metal such as Sn(Bu)₃ or Zn, under standard Suzuki conditions (e.g., in the presence of a palladium catalyst, such as tetrakis(triphenylphosphine)palladium(0) and a base (e.g., a carbonate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium(II)), to give compound 1-10. Cyclic urea 1-10 can be converted to halide 1-11, where X³ is a halogen (e.g., Cl, Br, or I), under standard conditions (e.g., in the presence of LDA or alkyllithium, 1,2-dibromotetrachloroethane in the case of bromination). Halide 1-11 can be coupled with compound 1-12, where M² is a boronic acid, boronate ester, potassium trifluoroborate, or an appropriately substituted metal such as Sn(Bu)₃ or Zn, under standard Suzuki conditions (e.g., in the presence of a palladium catalyst, such as tetrakis(triphenylphosphine)palladium(0) and a base (e.g., a carbonate base)) or standard Stille conditions (e.g., in the presence of a palladium(0) catalyst, such as tetrakis(triphenylphosphine)palladium(0)) or standard Negishi conditions (e.g., in the presence of a palladium catalyst, such as tetrakis(triphenylphosphine)palladium(0) or [1,1′-bis(diphenylphosphino)ferrocene]dichloropalladium(II)), to give compound 1-13. Compound 1-13 can be deprotected under standard conditions (e.g., NaOH when P=phenylsulfonyl) to give compounds of Formula I.

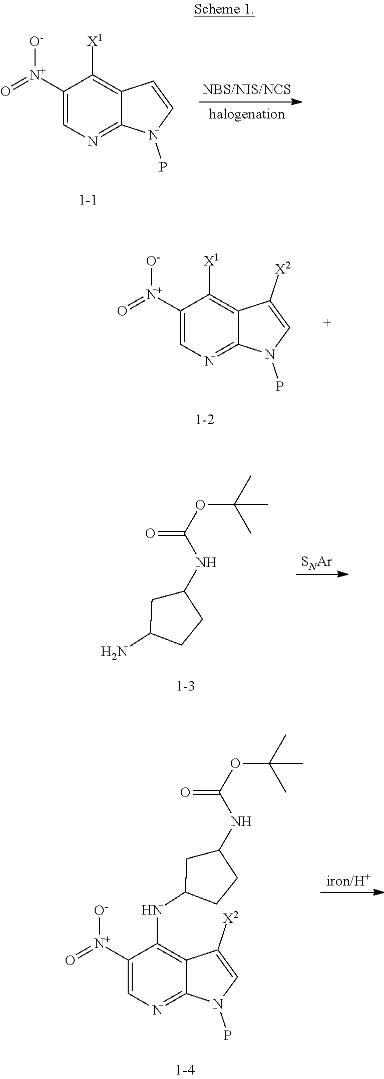

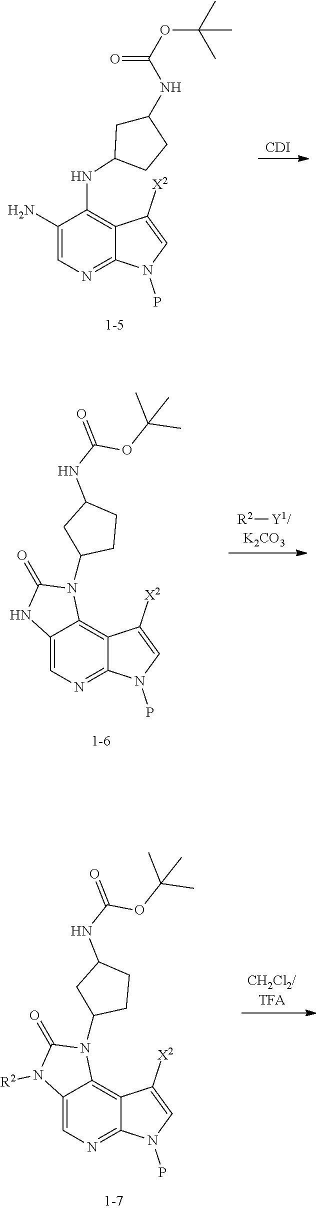

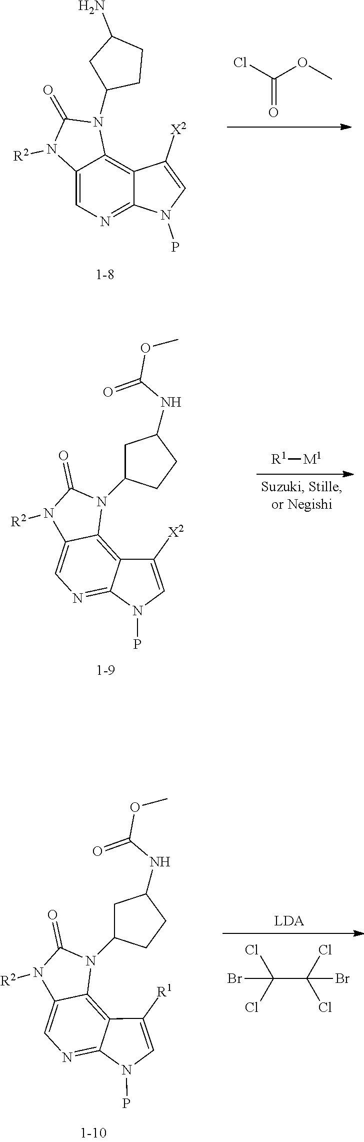

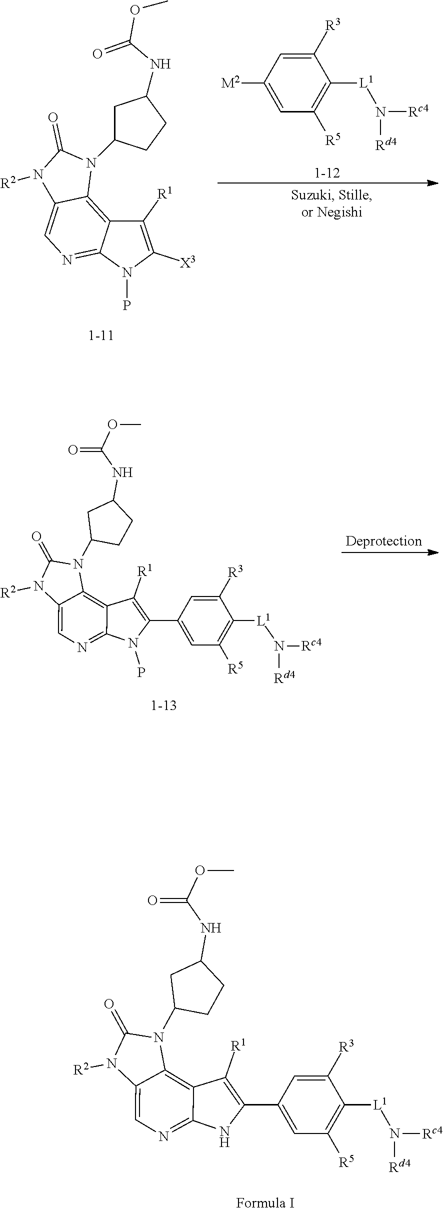

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

The expressions, “ambient temperature” or “room temperature” or “rt” as used herein, are understood in the art, and refer generally to a temperature, e.g., a reaction temperature, that is about the temperature of the room in which the reaction is carried out, for example, a temperature from about 20° C. to about 30° C.

Preparation of compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### Methods of Use

The compounds described herein can inhibit the activity of the V617F variant of the protein-tyrosine kinase JAK2 (i.e., “V617F” or “JAK2V617F”). Compounds which inhibit V617F are useful in providing a means of preventing the growth or inducing apoptosis in tumors, particularly by inhibiting angiogenesis. It is therefore anticipated that the compounds of the disclosure are useful in treating or preventing proliferative disorders such as cancers. In particular tumors with activating mutants of receptor tyrosine kinases or upregulation of receptor tyrosine kinases may be particularly sensitive to the inhibitors.

As disclosed herein, the compounds of the invention exhibit unexpectedly improved properties (e.g., improved potency, PK properties and safety profiles) compared to compounds disclosed in International Publication No.: WO 2022/006457, the disclosure of which is incorporated herein by reference in its entirety. The following compounds from WO 2022/006457 are provided herein as Comparative Examples A-F:

| Comparative | |
|---|---|
| Example | Structure |
| Comparative Example A | 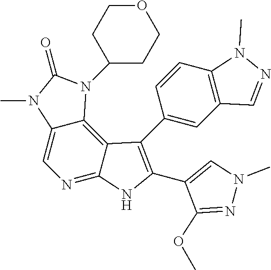 |
| Comparative Example B | 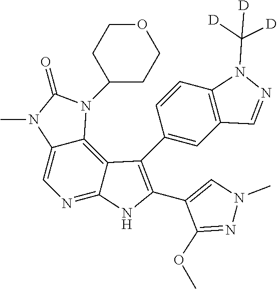 [SMILES: [2H]C([2H])([2H])n1ncc2cc(-c3c(-c4cn(C)nc4OC)[nH]c4ncc5c(c34)n(C3CCOCC3)c(=O)n5C)ccc21] |
| Comparative Example C | 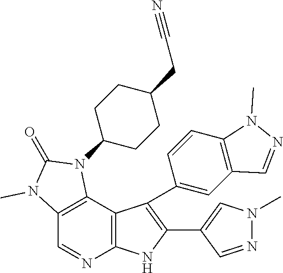 |
| Comparative Example D | 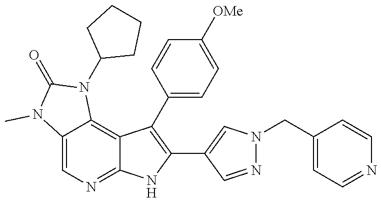 [SMILES: COc1ccc(-c2c(-c3cnn(Cc4ccncc4)c3)[nH]c3ncc4c(c23)n(C2CCCC2)c(=O)n4C)cc1] |
| Comparative Exampel E | 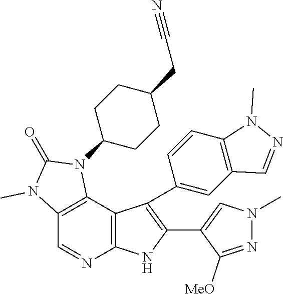 |
| Comparative Example F | 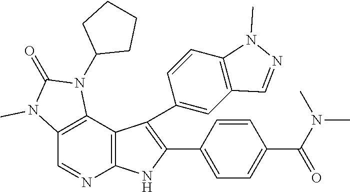 |
| Comparative Example G | 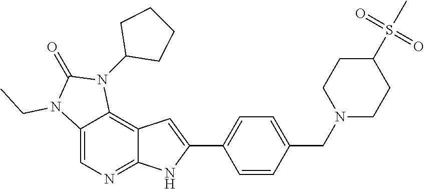 [SMILES: CCn1c(=O)n(C2CCCC2)c2c3cc(-c4ccc(CN5CCC(S(C)(=O)=O)CC5)cc4)[nH]c3ncc21] |
| Comparative Example H | 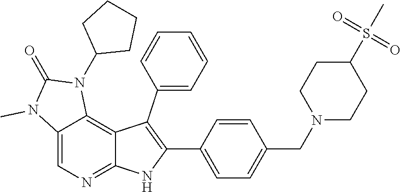 [SMILES: Cn1c(=O)n(C2CCCC2)c2c3c(-c4ccccc4)c(-c4ccc(CN5CCC(S(C)(=O)=O)CC5)cc4)[nH]c3ncc21] |
| Comparative Example I | 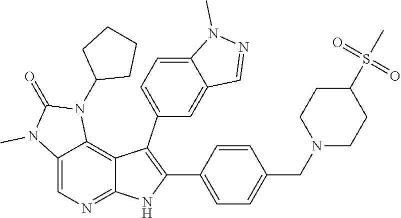 [SMILES: Cn1ncc2cc(-c3c(-c4ccc(CN5CCC(S(C)(=O)=O)CC5)cc4)[nH]c4ncc5c(c34)n(C3CCCC3)c(=O)n5C)ccc21] |

In certain embodiments, the disclosure provides a method for treating a V617F-related disorder in a patient in need thereof, comprising the step of administering to said patient a compound of the disclosure, or a pharmaceutically acceptable composition thereof.

Myeloproliferative diseases (MPD) are multipotent hematopoietic stem cell disorders characterized by excess production of various blood cells. MPNs include polycythemia vera (PV), essential thrombocythemia (ET), and idiopathic myelofibrosis (IMF). JAK2 V617F mutation is reported in about 95% of patients with PV, in 35% to 70% of patients with ET, and 50% of patients with IMF. Also, JAK2 exon 12 mutations are detected in some of the V617F-negative PV patients (Ma et al., J. Mol. Diagn., 11:49-53, 2009). In some embodiments, the compounds of the disclosure can be useful in the treatment of myeloproliferative disorders (e.g., myeloproliferative neoplasms) in a patient in need thereof, such as polycythemia vera, essential thrombocythemia, myelofibrosis with myeloid metaplasia (MMM), primary myelofibrosis (PMF), chronic myelogenous leukemia (CML), chronic myelomonocytic leukemia (CMML), hypereosinophilic syndrome (HES), systemic mast cell disease (SMCD), and the like.

In some embodiments, the myeloproliferative disorder is a myeloproliferative neoplasm.

In some embodiments, the myeloproliferative disorder is myelofibrosis (e.g., primary myelofibrosis (PMF) or post polycythemia vera/essential thrombocythemia myelofibrosis (Post-PV/ET MF)).

In some embodiments, the myeloproliferative disorder is primary myelofibrosis (PMF).

In some embodiments, the myeloproliferative disorder is post-essential thrombocythemia myelofibrosis (Post-ET MF).

In some embodiments, the myeloproliferative disorder is post polycythemia vera myelofibrosis (Post-PV MF).

In some embodiments, the myeloproliferative disorder is selected from primary myelofibrosis (PMF), polycythemia vera (PV), and essential thrombocythemia (ET).

In some embodiments, the myeloproliferative neoplasm is primary myelofibrosis (PMF).

In some embodiments, the myeloproliferative neoplasm is polycythemia vera (PV).

In some embodiments, the myeloproliferative neoplasm is essential thrombocythemia (ET).

Myeloproliferative diseases include disorders of a bone marrow or lymph node-derived cell type, such as a white blood cell. A myeloproliferative disease can manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

For example, the compounds of the disclosure are useful in the treatment of cancer. Example cancers include bladder cancer (e.g., urothelial carcinoma, squamous cell carcinoma, adenocarcinoma), breast cancer (e.g., hormone R positive, triple negative), cervical cancer, colorectal cancer, cancer of the small intestine, colon cancer, rectal cancer, cancer of the anus, endometrial cancer, gastric cancer (e.g., gastrointestinal stromal tumors), head and neck cancer (e.g., cancers of the larynx, hypopharynx, nasopharynx, oropharynx, lips, and mouth, squamous head and neck cancers), kidney cancer (e.g., renal cell carcinoma, urothelial carcinoma, sarcoma, Wilms tumor), liver cancer (e.g., hepatocellular carcinoma, cholangiocellular carcinoma (e.g., intrahepatic, hilar or perihilar, distal extrahepatic), liver angiosarcoma, hepatoblastoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, vulvar cancer, esophageal cancer, gall bladder cancer, pancreatic cancer (e.g. exocrine pancreatic carcinoma), stomach cancer, thyroid cancer, parathyroid cancer, neuroendocrine cancer (e.g., pheochromocytoma, Merkel cell cancer, neuroendocrine carcinoma), skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, acute myeloid leukemia (AML), B-cell lymphoma, cutaneous T-cell lymphoma, acute myelogenous leukemia, Hodgkin's or non-Hodgkin's lymphoma, myeloproliferative neoplasms (e.g., 8p11 myeloproliferative syndrome, polycythemia vera (PV), essential thrombocythemia (ET), and primary myelofibrosis (PMF)), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphomas, and Burkitt's lymphoma.

In certain embodiments, provided herein is a method of treating cancer comprising administering to a patient in need thereof a therapeutically effect amount of a compound of the disclosure. In certain embodiments, the cancer is selected from T lymphoblastic lymphoma, glioblastoma, melanoma, rhabdosarcoma, lymphosarcoma, and osteosarcoma.

Other cancers treatable with the compounds of the disclosure include tumors of the eye, glioblastoma, melanoma, leiomyosarcoma, and urothelial carcinoma (e.g., ureter, urethra, bladder, urachus).

The compounds of the disclosure can also be useful in the inhibition of tumor metastases.

In some embodiments, the compounds of the disclosure as described herein can be used to treat Alzheimer's disease, HIV, or tuberculosis.

In some embodiments, the compounds of the disclosure can be useful in the treatment of myelodysplastic syndrome (MDS) in a patient in need thereof. In some embodiments, said patient having the myelodysplastic syndrome (MDS) is red blood cell transfusion dependent.

As used herein, myelodysplastic syndromes are intended to encompass heterogeneous and clonal hematopoietic disorders that are characterized by ineffective hematopoiesis on one or more of the major myeloid cell lineages. Myelodysplastic syndromes are associated with bone marrow failure, peripheral blood cytopenias, and a propensity to progress to acute myeloid leukemia (AML). Moreover, clonal cytogenetic abnormalities can be detected in about 50% of cases with MDS. In 1997, The World Health Organization (WHO) in conjunction with the Society for Hematopathology (SH) and the European Association of Hematopathology (EAHP) proposed new classifications for hematopoietic neoplasms (Harris, et al., J Clin Oncol 1999; 17:3835-3849; Vardiman, et al., Blood 2002; 100:2292-2302). For MDS, the WHO utilized not only the morphologic criteria from the French-American-British (FAB) classification but also incorporated available genetic, biologic, and clinical characteristics to define subsets of MDS (Bennett, et al., Br. J. Haematol. 1982; 51:189-199). In 2008, the WHO classification of MDS (Table 1) was further refined to allow precise and prognostically relevant subclassification of unilineage dysplasia by incorporating new clinical and scientific information (Vardiman, et al., Blood 2009; 114:937-951; Swerdlow, et al., WHO Classification of Tumours of Haematopoietic and Lymphoid Tissues. 4th Edition. Lyon France: IARC Press; 2008:88-103; Bunning and Germing, “Myelodysplastic syndromes/neoplasms” in Chapter 5, Swerdlow, et al, eds. WHO Classification of Tumours of Haematopoietic and Lymphoid Tissues. (ed. 4th edition): Lyon, France: IARC Press; 2008:88-103).

TABLE 1
2008 WHO Classification for De Novo Myelodysplastic Syndrome

| Subtype | Blood | Bone Marrow |
|---|---|---|
| Refractory cytopenia with | Single or Bicytopenia | Dysplasia in ≥10% of 1 cell |
| unilineage dysplasia | | line, <5% blasts |
| (RCUD) | | |
| Refractory anemia with | Anemia, no blasts | ≥15% of erythroid precursors |
| ring sideroblasts (RARS) | | w/ring sideroblasts, erythroid |
| | | dysplasia only, <5% blasts |
| Refractory cytopenia with | Cytopenia(s), <1 × | Dysplasia in ≥10% of cells in |
| multilineage dysplasia | 10⁹/L monocytes | ≥2 hematopoietic lineages, |
| | | ±15% ring sideroblasts, <5% |
| | | blasts |
| Refractory anemia with | Cytopenia(s), ≤2% to | Unilineage or multilineage |
| excess blasts-1 (RAEB-1) | 4% blasts, <1 × 10⁹/L | dysplasia, No Auer rods, 5% to |
| | monocytes | 9% blasts |
| Refractory anemia with | Cytopenia(s), ≤5% to | Unilineage or multilineage |
| excess blasts-2 (RAEB-2) | 19% blasts, <1 × 10⁹/L | dysplasia, ±Auer rods, 10% to |
| | monocytes | 19% blasts |
| Myelodysplastic | Cytopenias | Unilineage or no dysplasia but |
| syndrome, unclassified | | characteristic MDS |
| (MDS-U) | | cytogenetics, <5% blasts |
| MDS associated with | Anemia, platelets | Unilineage erythroid. Isolated |
| isolated del(5q) | normal or increased | del(5q), <5% blasts |

In some embodiments, the myelodysplastic syndrome is refractory cytopenia with unilineage dysplasia (RCUD).

In some embodiments, the myelodysplastic syndrome is refractory anemia with ring sideroblasts (RARS).

In some embodiments, the myelodysplastic syndrome is refractory anemia with ring sideroblasts associated with thrombocytosis (RARS-T).

In some embodiments, the myelodysplastic syndrome is refractory cytopenia with multilineage dysplasia.

In some embodiments, the myelodysplastic syndrome is refractory anemia with excess blasts-1 (RAEB-1).

In some embodiments, the myelodysplastic syndrome is refractory anemia with excess blasts-2 (RAEB-2).

In some embodiments, the myelodysplastic syndrome is myelodysplastic syndrome, unclassified (MDS-U).

In some embodiments, the myelodysplastic syndrome is myelodysplastic syndrome associated with isolated del(5q).

In some embodiments, the myelodysplastic syndrome is refractory to erythropoiesis-stimulating agents.

In some embodiments, the compounds of the disclosure can be useful in the treatment of myeloproliferative disorder/myelodysplastic overlap syndrome (MPD/MDS overlap syndrome).

In some embodiments, the compounds of the disclosure can be useful in the treatment of leukemia.

In some embodiments, the compounds of the disclosure can be useful in the treatment of acute myeloid leukemia (AML).

In addition to oncogenic neoplasms, the compounds of the disclosure can be useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes.

The compounds provided herein may further be useful in the treatment of fibrotic diseases, such as where a disease symptom or disorder is characterized by fibrosis. Example fibrotic diseases include liver cirrhosis, glomerulonephritis, pulmonary fibrosis, systemic fibrosis, rheumatoid arthritis, and wound healing.

In some embodiments, the compounds provided herein can be used in the treatment of a hypophosphatemia disorder such as, for example, X-linked hypophosphatemic rickets, autosomal recessive hypophosphatemic rickets, and autosomal dominant hypophosphatemic rickets, or tumor-induced osteromalacia.

In some embodiments, provided herein is a method of increasing survival or progression-free survival in a patient, comprising administering a compound provided herein to the patient. In some embodiments, the patient has cancer. In some embodiments, the patient has a disease or disorder described herein. As used herein, progression-free survival refers to the length of time during and after the treatment of a solid tumor that a patient lives with the disease but it does not get worse. Progression-free survival can refer to the length of time from first administering the compound until the earlier of death or progression of the disease. Progression of the disease can be defined by RECIST v. 1.1 (Response Evaluation Criteria in Solid Tumors), as assessed by an independent centralized radiological review committee. In some embodiments, administering of the compound results in a progression free survival that is greater than about 1 month, about 2 months, about 3 months, about 4 months, about 5 months, about 6 months, about 8 months, about 9 months, about 12 months, about 16 months, or about 24 months. In some embodiments, the administering of the compound results in a progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months. In some embodiments, the administering of the compound results in an increase of progression free survival that is at least about 1 month, about 2 months, about 3 months, about 4 months, about 5 months, about 6 months, about 8 months, about 9 months, or about 12 months; and less than about 24 months, about 16 months, about 12 months, about 9 months, about 8 months, about 6 months, about 5 months, about 4 months, about 3 months, or about 2 months.

The present disclosure further provides a compound described herein, or a pharmaceutically acceptable salt thereof, for use in any of the methods described herein.

The present disclosure further provides use of a compound described herein, or a pharmaceutically acceptable salt thereof, for the preparation of a medicament for use in any of the methods described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a V617F variant with a compound described herein includes the administration of a compound described herein to an individual or patient, such as a human, having a V617F variant, as well as, for example, introducing a compound described herein into a sample containing a cellular or purified preparation containing the V617F variant.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent such as an amount of any of the solid forms or salts thereof as disclosed herein that elicits the biological or medicinal response in a tissue, system, animal, individual or human that is being sought by a researcher, veterinarian, medical doctor or other clinician. An appropriate “effective” amount in any individual case may be determined using techniques known to a person skilled in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically-acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

As used herein, the term “treating” or “treatment” refers to inhibiting the disease; for example, inhibiting a disease, condition or disorder in an individual who is experiencing or displaying the pathology or symptomatology of the disease, condition or disorder (i.e., arresting further development of the pathology and/or symptomatology) or ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

It is appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination.

### Combination Therapies

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with compounds described herein for treatment or prevention of V617F-associated diseases, disorders or conditions, or diseases or conditions as described herein. The agents can be combined with the present compounds in a single dosage form, or the agents can be administered simultaneously or sequentially as separate dosage forms.

Compounds described herein can be used in combination with one or more other kinase inhibitors for the treatment of diseases, such as cancer, that are impacted by multiple signaling pathways. For example, a combination can include one or more inhibitors of the following kinases for the treatment of cancer: Akt1, Akt2, Akt3, TGF-βR, Pim, PKA, PKG, PKC, CaM-kinase, phosphorylase kinase, MEKK, ERK, MAPK, mTOR, EGFR, HER2, HER3, HER4, INS—R, IGF-1R, IR—R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. Additionally, the solid forms of the inhibitor as described herein can be combined with inhibitors of kinases associated with the PIK3/Akt/mTOR signaling pathway, such as PI3K, Akt (including Akt1, Akt2 and Akt3) and mTOR kinases.

In some embodiments, compounds described herein can be used in combination with one or more inhibitors of the enzyme or protein receptors such as HPK1, SBLB, TUT4, A2A/A2B, CD19, CD47, CDK2, STING, ALK2, LIN28, ADAR1, MAT2a, RIOK1, HDAC8, WDR5, SMARCA2, and DCLK1 for the treatment of diseases and disorders. Exemplary diseases and disorders include cancer, infection, inflammation and neurodegenerative disorders.

In some embodiments, compounds described herein can be used in combination with a therapeutic agent that targets an epigenetic regulator. Examples of epigenetic regulators include bromodomain inhibitors, the histone lysine methyltransferases, histone arginine methyl transferases, histone demethylases, histone deacetylases, histone acetylases, and DNA methyltransferases. Histone deacetylase inhibitors include, e.g., vorinostat.

For treating cancer and other proliferative diseases, compounds described herein can be used in combination with targeted therapies, including JAK kinase inhibitors (ruxolitinib, additional JAK1/2 and JAK1-selective, baricitinib or itacitinib), Pim kinase inhibitors (e.g., LGH447, INCB053914 and SGI-1776), PI3 kinase inhibitors including PI3K-delta selective and broad spectrum PI3K inhibitors (e.g., parsaclisib and INCB50797), PI3K-gamma inhibitors such as PI3K-gamma selective inhibitors, MEK inhibitors, CSFIR inhibitors (e.g., PLX3397 and LY3022855), TAM receptor tyrosine kinases inhibitors (Tyro-3, Ax1, and Mer; e.g., INCB81776), angiogenesis inhibitors, interleukin receptor inhibitors, Cyclin Dependent kinase inhibitors, BRAF inhibitors, mTOR inhibitors, proteasome inhibitors (Bortezomib, Carfilzomib), HDAC-inhibitors (panobinostat, vorinostat), DNA methyl transferase inhibitors, dexamethasone, bromo and extra terminal family members inhibitors (for example, bromodomain inhibitors or BET inhibitors, such as OTX015, CPI-0610, INCB54329 or INCB57643), LSD1 inhibitors (e.g., GSK2979552, INCB59872 and INCB60003), arginase inhibitors (e.g., INCB1158), indoleamine 2,3-dioxygenase inhibitors (e.g., epacadostat, NLG919 or BMS-986205), PARP inhibiors (e.g., olaparib or rucaparib), and inhibitors of BTK such as ibrutinib.

For treating cancer and other proliferative diseases, compounds described herein can be used in combination with chemotherapeutic agents, agonists or antagonists of nuclear receptors, or other anti-proliferative agents. Compounds described herein can also be used in combination with a medical therapy such as surgery or radiotherapy, e.g., gamma-radiation, neutron beam radiotherapy, electron beam radiotherapy, proton therapy, brachytherapy, and systemic radioactive isotopes.

Examples of suitable chemotherapeutic agents include any of: abarelix, abiraterone, afatinib, aflibercept, aldesleukin, alemtuzumab, alitretinoin, allopurinol, altretamine, amidox, amsacrine, anastrozole, aphidicolon, arsenic trioxide, asparaginase, axitinib, azacitidine, bevacizumab, bexarotene, baricitinib, bendamustine, bicalutamide, bleomycin, bortezombi, bortezomib, brivanib, buparlisib, busulfan intravenous, busulfan oral, calusterone, camptosar, capecitabine, carboplatin, carmustine, cediranib, cetuximab, chlorambucil, cisplatin, cladribine, clofarabine, crizotinib, cyclophosphamide, cytarabine, dacarbazine, dacomitinib, dactinomycin, dalteparin sodium, dasatinib, dactinomycin, daunorubicin, decitabine, degarelix, denileukin, denileukin diftitox, deoxycoformycin, dexrazoxane, didox, docetaxel, doxorubicin, droloxafine, dromostanolone propionate, eculizumab, enzalutamide, epidophyllotoxin, epirubicin, epothilones, erlotinib, estramustine, etoposide phosphate, etoposide, exemestane, fentanyl citrate, filgrastim, floxuridine, fludarabine, fluorouracil, flutamide, fulvestrant, gefitinib, gemcitabine, gemtuzumab ozogamicin, goserelin acetate, histrelin acetate, ibritumomab tiuxetan, idarubicin, idelalisib, ifosfamide, imatinib mesylate, interferon alfa 2a, irinotecan, lapatinib ditosylate, lenalidomide, letrozole, leucovorin, leuprolide acetate, levamisole, lonafarnib, lomustine, meclorethamine, megestrol acetate, melphalan, mercaptopurine, methotrexate, methoxsalen, mithramycin, mitomycin C, mitotane, mitoxantrone, nandrolone phenpropionate, navelbene, necitumumab, nelarabine, neratinib, nilotinib, nilutamide, niraparib, nofetumomab, oserelin, oxaliplatin, paclitaxel, pamidronate, panitumumab, panobinostat, pazopanib, pegaspargase, pegfilgrastim, pemetrexed disodium, pentostatin, pilaralisib, pipobroman, plicamycin, ponatinib, porfimer, prednisone, procarbazine, quinacrine, ranibizumab, rasburicase, regorafenib, reloxafine, revlimid, rituximab, rucaparib, ruxolitinib, sorafenib, streptozocin, sunitinib, sunitinib maleate, tamoxifen, tegafur, temozolomide, teniposide, testolactone, tezacitabine, thalidomide, thioguanine, thiotepa, tipifarnib, topotecan, toremifene, tositumomab, trastuzumab, tretinoin, triapine, trimidox, triptorelin, uracil mustard, valrubicin, vandetanib, vinblastine, vincristine, vindesine, vinorelbine, vorinostat, veliparib, talazoparib, and zoledronate.

In some embodiments, compounds described herein can be used in combination with immune checkpoint inhibitors. Exemplary immune checkpoint inhibitors include inhibitors against immune checkpoint molecules such as CD27, CD28, CD40, CD122, CD96, CD73, CD47, OX40, GITR, CSFIR, JAK, PI3K delta, PI3K gamma, TAM, arginase, CD137 (also known as 4-1BB), ICOS, A2AR, B7-H3, B7-H4, BTLA, CTLA-4, LAG3 (e.g., INCAGN2385), TIM3 (e.g., INCB2390), VISTA, PD-1, PD-L1 and PD-L2. In some embodiments, the immune checkpoint molecule is a stimulatory checkpoint molecule selected from CD27, CD28, CD40, ICOS, OX40 (e.g., INCAGN1949), GITR (e.g., INCAGN1876) and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is a small molecule PD-L1 inhibitor. In some embodiments, the small molecule PD-L1 inhibitor has an IC50 less than 1 μM, less than 100 nM, less than 10 nM or less than 1 nM in a PD-L1 assay described in US Patent Publication Nos. US20170107216, US20170145025, US 20170174671, US20170174679, US20170320875, US20170342060, US20170362253, and US20180016260, each of which is incorporated by reference in its entirety for all purposes.

In some embodiments, the inhibitor of an immune checkpoint molecule is an inhibitor of PD-1, e.g., an anti-PD-1 monoclonal antibody. In some embodiments, the anti-PD-1 monoclonal antibody is retifanlimab (also known as MGA012), nivolumab, pembrolizumab (also known as MK-3475), pidilizumab, SHR-1210, PDR001, ipilumimab or AMP-224. In some embodiments, the anti-PD-1 monoclonal antibody is nivolumab or pembrolizumab. In some embodiments, the anti-PD1 antibody is pembrolizumab. In some embodiments, the anti-PD1 antibody is nivolumab. In some embodiments, the anti-PD-1 monoclonal antibody is retifanlimab. In some embodiments, the anti-PD1 antibody is SHR-1210. Other anti-cancer agent(s) include antibody therapeutics such as 4-1BB (e.g. urelumab, utomilumab.

In some embodiments, the compounds of the disclosure can be used in combination with INCB086550.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, or INCAGN2385.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of GITR, e.g., an anti-GITR antibody. In some embodiments, the anti-GITR antibody is TRX518, MK-4166, INCAGN1876, MK-1248, AMG228, BMS-986156, GWN323, or MEDI1873.

In some embodiments, the inhibitor of an immune checkpoint molecule is an agonist of OX40, e.g., OX40 agonist antibody or OX40L fusion protein. In some embodiments, the anti-OX40 antibody is MEDI0562, MOXR-0916, PF-04518600, GSK3174998, or BMS-986178. In some embodiments, the OX40L fusion protein is MEDI6383.

In some embodiments, the inhibitor of an immune checkpoint molecule is an inhibitor of CD20, e.g., an anti-CD20 antibody. In some embodiments, the anti-CD20 antibody is obinutuzumab or rituximab.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

In some embodiments, the compounds described herein can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Suitable antiviral agents contemplated for use in combination with compounds of the present disclosure can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No.11607.

Suitable agents for use in combination with compounds described herein for the treatment of cancer include chemotherapeutic agents, targeted cancer therapies, immunotherapies or radiation therapy. Compounds described herein may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds described herein. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

The compounds described herein may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with inhibitors described herein. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds described herein include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with compounds described herein. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds described herein. In some embodiments, the JAK inhibitor is selective for JAK1 over JAK2 and JAK3.

Other suitable agents for use in combination with compounds described herein include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide, ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with compounds described herein include steroids including 17 alpha-ethinylestradiol, diethylstilbestrol, testosterone, prednisone, fluoxymesterone, methylprednisolone, methyltestosterone, prednisolone, triamcinolone, chlorotrianisene, hydroxyprogesterone, aminoglutethimide, and medroxyprogesteroneacetate.

Other suitable agents for use in combination with compounds described herein include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds described herein may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (e.g., vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel, mithramycin, deoxycoformycin, mitomycin-C, L-asparaginase, interferons (especially IFN-a), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB, PD-L1 and PD-1 antibodies, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of the present disclosure can be used in combination with bone marrow transplant for the treatment of a variety of tumors of hematopoietic origin (see e.g., U.S. Pat. Nos. 9,233,985, 10,065,974, 10,287,303, 8,524,867, the disclosures of which are incorporated by reference herein in their entireties).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), more usually about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating V617F in tissue samples, including human, and for identifying V617F inhibitors by binding of a labeled compound. Substitution of one or more of the atoms of the compounds of the present disclosure can also be useful in generating differentiated ADME (Adsorption, Distribution, Metabolism and Excretion.) Accordingly, the present disclosure includes V617F assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms to allow the compound to be deuterated (e.g., one or more hydrogen atoms of a C₁₋₆ alkyl group of Formula I can be optionally substituted with deuterium atoms, such as —CD₃ (i.e., trideuteromethyl) being substituted for —CH₃). In some embodiments, alkyl groups of the disclosed Formulas (e.g., Formula I) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, 1-6, 1-8, 1-10, 1-12, 1-14, 1-16, 1-18, or 1-20 deuterium atoms. In some embodiments, all of the hydrogen atoms in a compound can be replaced or substituted by deuterium atoms.

In some embodiments, each hydrogen atom of the compounds provided herein, such as hydrogen atoms attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is optionally replaced by deuterium atoms.

In some embodiments, each hydrogen atom of the compounds provided herein, such as hydrogen atoms to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, is replaced by deuterium atoms (i.e., the alkyl, alkenyl, alkynyl, aryl, phenyl, cycloalkyl, heterocycloalkyl, or heteroaryl substituents, or —C₁₋₄ alkyl-, alkylene, alkenylene, and alkynylene linking groups are perdeuterated).

In some embodiments, 1, 2, 3, 4, 5, 6, 7, 8, 9, 10, 11, or 12 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene, and alkynylene linking groups, as described herein, are optionally replaced by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

In some embodiments, the compound provided herein (e.g., the compound of any of Formulas I-IVc), or a pharmaceutically acceptable salt thereof, comprises at least one deuterium atom.

In some embodiments, the compound provided herein (e.g., the compound of any of Formulas I-IVc), or a pharmaceutically acceptable salt thereof, comprises two or more deuterium atoms.

In some embodiments, the compound provided herein (e.g., the compound of any of Formulas I-IVc), or a pharmaceutically acceptable salt thereof, comprises three or more deuterium atoms.

In some embodiments, for a compound provided herein (e.g., the compound of any of Formulas I-IVc), or a pharmaceutically acceptable salt thereof, all of the hydrogen atoms are replaced by deuterium atoms (i.e., the compound is “perdeuterated”).

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro V617F labeling and competition assays, compounds that incorporate ³H, ¹⁴C, ⁸²Br, ¹²⁵I, ¹³¹I or ³⁵S can be useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and an ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind V617F by monitoring its concentration variation when contacting with V617F, through tracking of the labeling. For example, a test compound (labeled) can be evaluated for its ability to reduce binding of another compound which is known to bind to V617F (i.e., standard compound). Accordingly, the ability of a test compound to compete with the standard compound for binding to V617F directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of V617F-associated diseases or disorders as described herein, which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Preparatory LC-MS purifications of some of the compounds prepared were performed on Waters mass directed fractionation systems. The basic equipment setup, protocols, and control software for the operation of these systems have been described in detail in the literature (see e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004)).

The compounds separated were typically subjected to analytical liquid chromatography mass spectrometry (LCMS) for purity analysis under the following conditions: Instrument=Agilent 1100 series, LC/MSD; Column: Waters Sunfire™ C₁₈ 5 μm, 2.1×50 mm, Buffers: mobile phase A: 0.025% TFA in water and mobile phase B: acetonitrile; gradient 2% to 80% B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:
pH=2 purifications: Waters Sunfire™ C₁₈ 5 μm, 30×100 mm or Waters XBridge™ C₁₈ 5 μm, 30×100 mm column, eluting with mobile phase A: 0.1% TFA (trifluoroacetic acid) in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).
pH=10 purifications: Waters XBridge™ C₁₈ 5 μm, 30×100 mm column, eluting with mobile phase A: 0.1% NH₄OH in water and mobile phase B: acetonitrile; the flow rate was 60 mL/minute, the separating gradient was optimized for each compound using the Compound Specific Method Optimization protocol as described in the literature (see e.g., “Preparative LCMS Purification: Improved Compound Specific Method Optimization”, K. Blom, B. Glass, R. Sparks, A. Combs, J. Comb. Chem., 6, 874-883 (2004)).

### Example 1. Methyl ((1R,3R)-3-(7-(3-fluoro-4-(methylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate

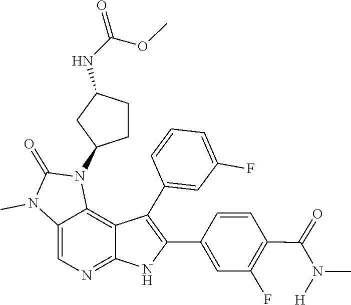

A procedure analogous to the one described in Example 25 was used to prepare the title compound with the exception that (3-fluorophenyl) boronic acid was used in step 7 and (3-fluoro-4-(methylcarbamoyl)phenyl) boronic acid was used in step 9. LCMS for C₃₀H₂₉F₂N₆O₄ (M+H)⁺: m/z=575.2; Found: 575.2.

### Example 2. Methyl ((1R,3R)-3-(7-(3-fluoro-4-(isopropylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate

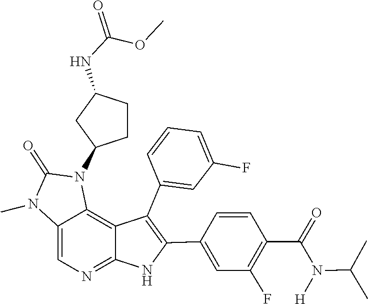 [SMILES: COC(=O)N[C@@H]1CC[C@@H](n2c(=O)n(C)c3cnc4[nH]c(-c5ccc(C(=O)NC(C)C)c(F)c5)c(-c5cccc(F)c5)c4c32)C1]

A procedure analogous to the one described in Example 1 was used to prepare the title compound. LCMS for C₃₂H₃₃F₂N₆O₄ (M+H)⁺: m/z=603.3; Found: 603.4.

### Example 3. Methyl ((1R,3R)-3-(7-(3,5-difluoro-4-(methylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate

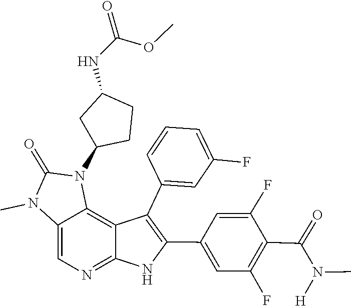

A procedure analogous to the one described in Example 1 was used to prepare the title compound. LCMS for C₃₀H₂₈F₃N₆O₄ (M+H)⁺: m/z=593.2; Found: 593.3.

### Example 4. Methyl ((1R,3R)-3-(7-(4-(cyclopropylcarbamoyl)-3,5-difluorophenyl)-8-(3-fluorophenyl)-3-methyl-2-oxo-3,6-dihydroimidazo[4,5-d]pyrrolo[2,3-b]pyridin-1(2H)-yl)cyclopentyl)carbamate

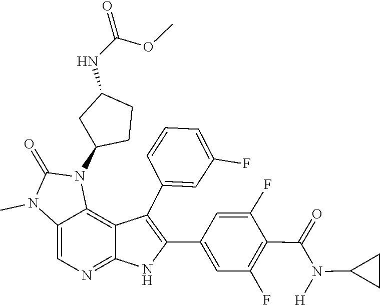 [SMILES: COC(=O)N[C@@H]1CC[C@@H](n2c(=O)n(C)c3cnc4[nH]c(-c5cc(F)c(C(=O)NC6CC6)c(F)c5)c(-c5cccc(F)c5)c4c32)C1]

A procedure analogous to the one described in Example 1 was used to prepare the title compound. LCMS for C₃₂H₃₀F₃N₆O₄ (M+H)⁺: m/z=619.2; Found: 619.4.

### Example 5. Methyl ((1R,3R)-3-(7-(4-(3-oxa-8-azabicyclo[3.2.1]octane-8-carbonyl)-3-methoxyphenyl)-8-(3-fluorophenyl)-3-methyl-2-oxo-3,6-dihydroimidazo[4,5-d]pyrrolo[2,3-b]pyridin-1(2H)-yl)cyclopentyl)carbamate

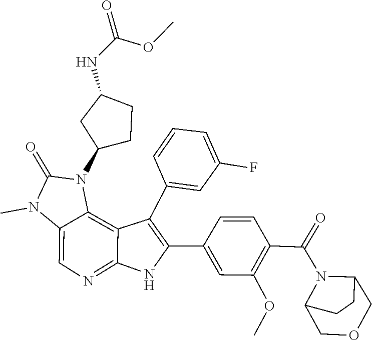 [SMILES: COC(=O)N[C@@H]1CC[C@@H](n2c(=O)n(C)c3cnc4[nH]c(-c5ccc(C(=O)N6C7CCC6COC7)c(OC)c5)c(-c5cccc(F)c5)c4c32)C1]

A procedure analogous to the one described in Example 1 was used to prepare the title compound. LCMS for C₃₆H₃₈FN₆O₆ (M+H)⁺: m/z=669.3; Found: 669.3.

### Example 6. Methyl ((1R,3R)-3-(7-(4-(ethylcarbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

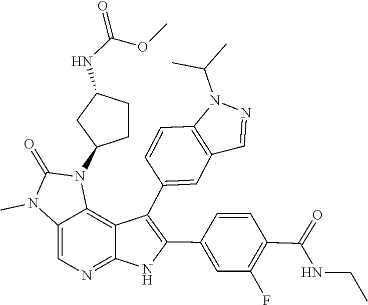 [SMILES: CCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1F]

A procedure analogous to the one described in Example 8 was used to prepare the title compound. LCMS for C₃₅H₃₈FN₈O₄ (M+H)⁺: m/z=653.3; Found: 653.4.

### Example 7. Methyl ((1R,3R)-3-(7-(3-fluoro-4-((3-(phenylamino)propyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

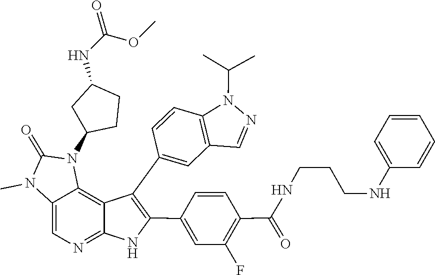 [SMILES: COC(=O)N[C@@H]1CC[C@@H](n2c(=O)n(C)c3cnc4[nH]c(-c5ccc(C(=O)NCCCNc6ccccc6)c(F)c5)c(-c5ccc6c(cnn6C(C)C)c5)c4c32)C1]

A procedure analogous to the one described in Example 8 was used to prepare the title compound. LCMS for C₄₂H₄₅FN₉O₄ (M+H)⁺: m/z=758.4; Found: 758.4.

### Example 8. Methyl ((1R,3R)-3-(7-(4-(cyclopropylcarbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

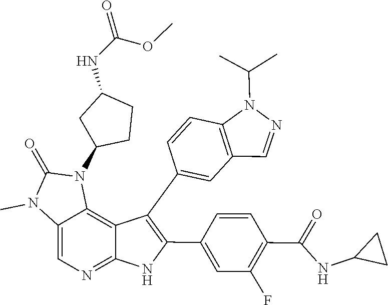 [SMILES: COC(=O)N[C@@H]1CC[C@@H](n2c(=O)n(C)c3cnc4[nH]c(-c5ccc(C(=O)NC6CC6)c(F)c5)c(-c5ccc6c(cnn6C(C)C)c5)c4c32)C1]

### Step 1. Methyl 2-fluoro-4-(8-(1-isopropyl-1H-indazol-5-yl)-1-((1R,3R)-3-((methoxycarbonyl)amino)cyclopentyl)-3-methyl-2-oxo-6-(phenylsulfonyl)-1,2,3,6-tetrahydroimidazo[4,5-d]pyrrolo[2,3-b]pyridin-7-yl)benzoate

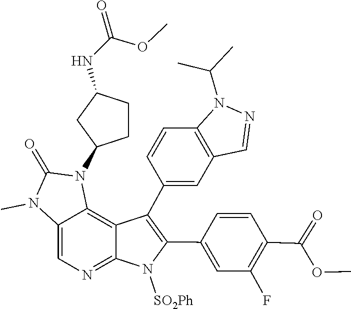

A mixture of (3-fluoro-4-(methoxycarbonyl)phenyl) boronic acid (58.8 mg, 0.297 mmol, Combi-Blocks BB-2719, [505083-04-5]), methyl ((1R,3R)-3-(7-bromo-8-(1-isopropyl-1H-indazol-5-yl)-3-methyl-2-oxo-6-(phenylsulfonyl)-3,6-dihydroimidazo[4,5-d]pyrrolo[2,3-b]pyridin-1(2H)-yl)cyclopentyl)carbamate (70 mg, 0.099 mmol, prepared using a procedure analogous to that described for Example 25, steps 1-8), PdCl₂ (dppf)-CH₂Cl₂ adduct (16.2 mg, 0.0200 mmol), and 1M K₂CO₃ (aq) (0.297 mL, 0.297 mmol) in 1,4-dioxane (2 mL) was degassed and purged with N₂ (g) several times prior to heating in a sealed vial at 110° C. After 5 h, the crude reaction mixture was diluted with EtOAc (20 mL) and filtered through a pad of Celite™. The filtrate was washed with brine, dried over N₂SO₄, filtered, and concentrated in-vacuo. The crude product was purified by CombiFlash chromatography (24 g silica gel column, eluting with 0-15% methanol/dichloromethane) to afford 70 mg of desired product (91% yield). LCMS for C₄₀H₃₉FN₇O₇S (M+H)⁺: m/z=780.3; Found: 780.4.

### Step 2. 2-Fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzoic acid

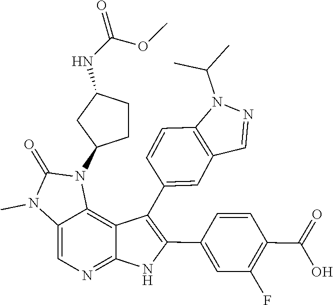 [SMILES: COC(=O)N[C@@H]1CC[C@@H](n2c(=O)n(C)c3cnc4[nH]c(-c5ccc(C(=O)O)c(F)c5)c(-c5ccc6c(cnn6C(C)C)c5)c4c32)C1]

A mixture of methyl 2-fluoro-4-(8-(1-isopropyl-1H-indazol-5-yl)-1-((1R,3R)-3-((methoxycarbonyl)amino)cyclopentyl)-3-methyl-2-oxo-6-(phenylsulfonyl)-1,2,3,6-tetrahydroimidazo[4,5-d]pyrrolo[2,3-b]pyridin-7-yl)benzoate (70 mg, 0.090 mmol) and lithium hydroxide monohydrate (38 mg, 0.90 mmol) in THF (1 mL), methanol (0.67 mL), and water (0.33 mL) was heated at 65° C. After 5 h, the reaction was diluted with H₂O (5 mL) and washed with ethyl acetate (2×5 mL) prior to neutralization with 1M HCl (aq) (0.89 mL, 0.89 mmol). The product partially precipitated out and was filtered off. The aqueous filtrate was extracted with DCM (4×5 mL), washed with brine, dried over N₂SO₄, filtered, and concentrated in-vacuo. The crude product was used in the next step without further purification. LCMS for C₃₃H₃₃FN₇O₅ (M+H)⁺: m/z=626.2; Found: 626.3.

### Step 3. methyl ((1R,3R)-3-(7-(4-(cyclopropylcarbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

To a solution of 2-fluoro-4-(8-(1-isopropyl-1H-indazol-5-yl)-1-((1R,3R)-3-((methoxycarbonyl)amino)cyclopentyl)-3-methyl-2-oxo-1,2,3,6-tetrahydroimidazo[4,5-d]pyrrolo[2,3-b]pyridin-7-yl)benzoic acid (10 mg, 0.016 mmol) and HATU (9.1 mg, 0.024 mmol) in DMF (0.5 mL) was added DIEA (0.011 mL, 0.064 mmol) and 1-(2-methoxyethyl)cyclopropan-1-amine, HCl (4.9 mg, 0.032 mmol). After stirring for 2 h, the reaction mixture was diluted with acetonitrile, filtered, and purified using prep-LCMS (XBridge C18 column, eluting with a gradient of acetonitrile/water containing 0.1% TFA, at flow rate of 60 mL/min) to afford the desired product. LCMS for C₃₆H₃₈FN₈O₄ (M+H)⁺: m/z=665.3; Found: 665.3.

### Example 9. Methyl ((1R,3R)-3-(7-(4-((2,2-difluorocyclopropyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

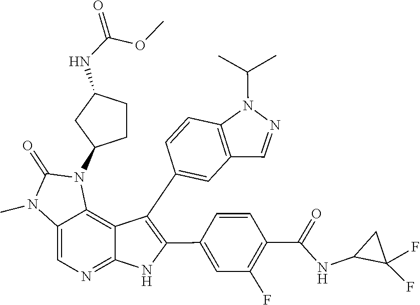 [SMILES: COC(=O)N[C@@H]1CC[C@@H](n2c(=O)n(C)c3cnc4[nH]c(-c5ccc(C(=O)NC6CC6(F)F)c(F)c5)c(-c5ccc6c(cnn6C(C)C)c5)c4c32)C1]

A procedure analogous to the one described in Example 8 was used to prepare the title compound. LCMS for C₃₆H₃₆F₃N₈O₄ (M+H)⁺: m/z=701.3; Found: 701.4.

### Example 10. Methyl ((1R,3R)-3-(7-(3-fluoro-4-((2-(trifluoromethyl)cyclopropyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

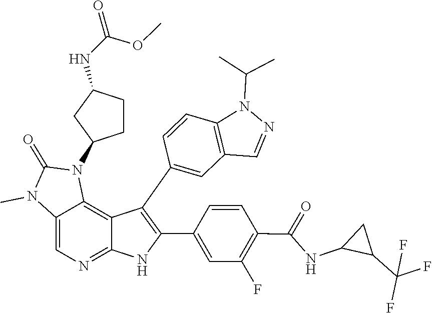 [SMILES: COC(=O)N[C@@H]1CC[C@@H](n2c(=O)n(C)c3cnc4[nH]c(-c5ccc(C(=O)NC6CC6C(F)(F)F)c(F)c5)c(-c5ccc6c(cnn6C(C)C)c5)c4c32)C1]

A procedure analogous to the one described in Example 8 was used to prepare the title compound. LCMS for C₃₇H₃₇F₄N₈O₄ (M+H)⁺: m/z=733.3; Found: 733.4.

### Example 11. Methyl ((1R,3R)-3-(7-(3-fluoro-4-((2,2,2-trifluoroethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

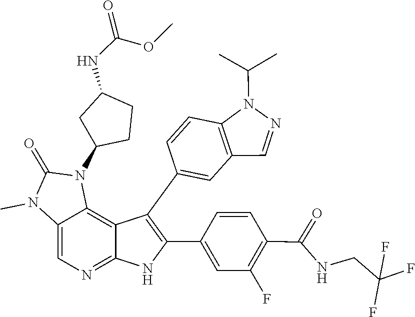 [SMILES: COC(=O)N[C@@H]1CC[C@@H](n2c(=O)n(C)c3cnc4[nH]c(-c5ccc(C(=O)NCC(F)(F)F)c(F)c5)c(-c5ccc6c(cnn6C(C)C)c5)c4c32)C1]

A procedure analogous to the one described in Example 8 was used to prepare the title compound. LCMS for C₃₅H₃₅F₄N₈O₄ (M+H)⁺: m/z=707.3; Found: 707.4.

### Example 12. Methyl ((1R,3R)-3-(7-(3-fluoro-4-(methylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

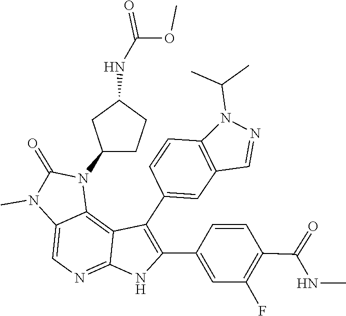

A procedure analogous to the one described in Example 1 was used to prepare the title compound. LCMS for C₃₄H₃₆FN₈O₄ (M+H)⁺: m/z=639.3; Found: 639.4.

### Example 13. Methyl ((1R,3R)-3-(7-(3-fluoro-4-((1-(trifluoromethyl)cyclopropyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

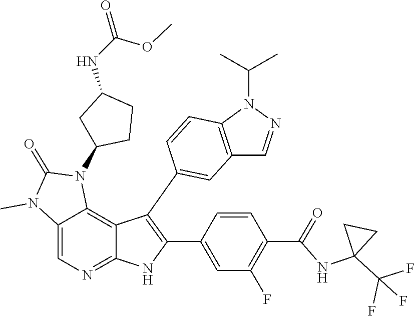 [SMILES: COC(=O)N[C@@H]1CC[C@@H](n2c(=O)n(C)c3cnc4[nH]c(-c5ccc(C(=O)NC6(C(F)(F)F)CC6)c(F)c5)c(-c5ccc6c(cnn6C(C)C)c5)c4c32)C1]

A procedure analogous to the one described in Example 8 was used to prepare the title compound. LCMS for C₃₇H₃₇F₄N₈O₄ (M+H)⁺: m/z=733.3; Found: 733.3.

### Example 14. Methyl ((1R,3R)-3-(7-(3-fluoro-4-((2-methoxyethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

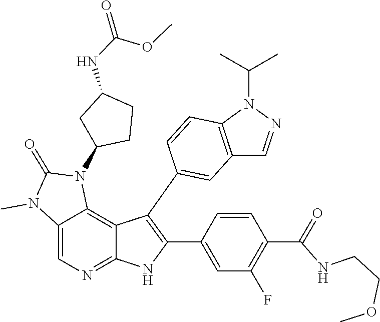 [SMILES: COCCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1F]

A procedure analogous to the one described in Example 8 was used to prepare the title compound. LCMS for C₃₆H₄₀FN₈O₅ (M+H)⁺: m/z=683.3; Found: 683.3.

### Example 15. Methyl ((1R,3R)-3-(7-(3-fluoro-4-((2-methoxyethyl)(methyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

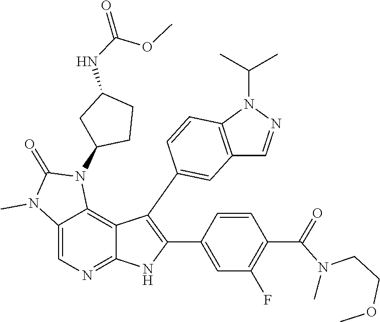 [SMILES: COCCN(C)C(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1F]

A procedure analogous to the one described in Example 8 was used to prepare the title compound. LCMS for C₃₇H₄₂FN₈O₅ (M+H)⁺: m/z=697.3; Found: 697.3.

### Example 16. Methyl ((1R,3R)-3-(7-(3-fluoro-4-(morpholine-4-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

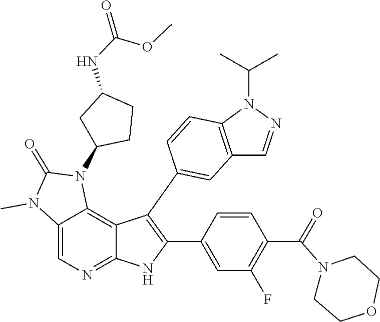 [SMILES: COC(=O)N[C@@H]1CC[C@@H](n2c(=O)n(C)c3cnc4[nH]c(-c5ccc(C(=O)N6CCOCC6)c(F)c5)c(-c5ccc6c(cnn6C(C)C)c5)c4c32)C1]

A procedure analogous to the one described in Example 8 was used to prepare the title compound. LCMS for C₃₇H₄₀FN₈O₅ (M+H)⁺: m/z=695.3; Found: 695.3.

### Example 17. Methyl ((1R,3R)-3-(7-(3-fluoro-4-(3-methoxyazetidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

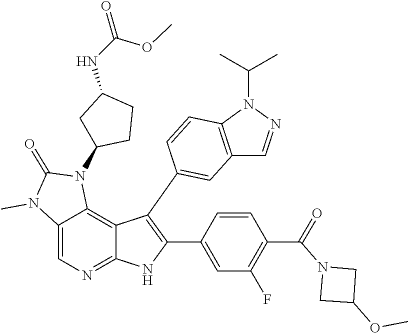

A procedure analogous to the one described in Example 8 was used to prepare the title compound. LCMS for C₃₇H₄₀FN₈O₅ (M+H)⁺: m/z=695.3; Found: 695.3.

### Example 18. Methyl ((1R,3R)-3-(7-(4-(ethylcarbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

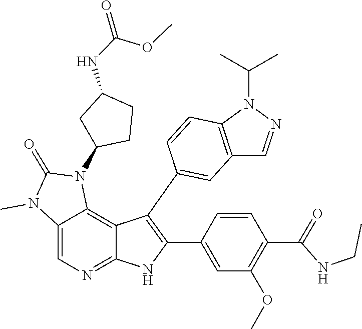 [SMILES: CCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1OC]

A procedure analogous to the one described in Example 8 was used to prepare the title compound. LCMS for C₃₆H₄₁N₈O₅ (M+H)⁺: m/z=665.3; Found: 665.3.

### Example 19. Methyl ((1R,3R)-3-(8-(1-isopropyl-1H-indazol-5-yl)-7-(4-((2-methoxyethyl)carbamoyl)phenyl)-3-methyl-2-oxo-3,6-dihydroimidazo[4,5-d]pyrrolo[2,3-b]pyridin-1(2H)-yl)cyclopentyl)carbamate

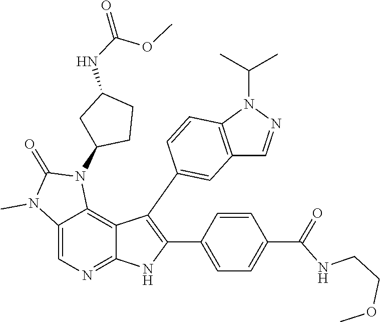 [SMILES: COCCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1]

A procedure analogous to the one described in Example 8 was used to prepare the title compound. LCMS for C₃₆H₄₁N₈O₅ (M+H)⁺: m/z=665.3; Found: 665.2.

### Example 20. Methyl ((1R,3R)-3-(8-(1-isopropyl-1H-indazol-5-yl)-3-methyl-7-(4-(morpholine-4-carbonyl)phenyl)-2-oxo-3,6-dihydroimidazo[4,5-d]pyrrolo[2,3-b]pyridin-1(2H)-yl)cyclopentyl)carbamate

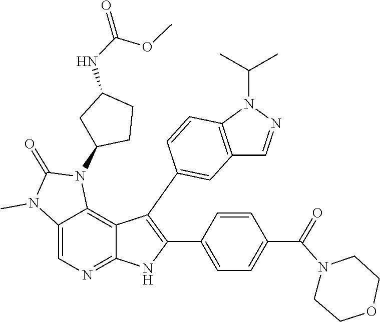 [SMILES: COC(=O)N[C@@H]1CC[C@@H](n2c(=O)n(C)c3cnc4[nH]c(-c5ccc(C(=O)N6CCOCC6)cc5)c(-c5ccc6c(cnn6C(C)C)c5)c4c32)C1]

A procedure analogous to the one described in Example 8 was used to prepare the title compound. LCMS for C₃₇H₄₁N₈O₅ (M+H)⁺: m/z=677.3; Found: 677.3.

### Example 21. Methyl ((1R,3R)-3-(8-(1-isopropyl-1H-indazol-5-yl)-3-methyl-7-(4-(methylcarbamoyl)phenyl)-2-oxo-3,6-dihydroimidazo[4,5-d]pyrrolo[2,3-b]pyridin-1(2H)-yl)cyclopentyl)carbamate

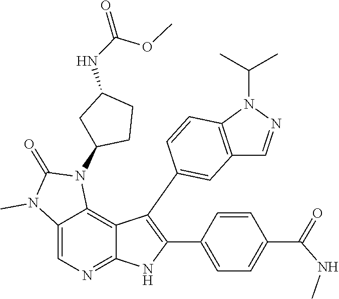 [SMILES: CNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1]

A procedure analogous to the one described in Example 8 was used to prepare the title compound. LCMS for C₃₄H₃₇N₈O₄ (M+H)⁺: m/z=621.3; Found: 621.2.

### Example 22. Methyl ((1R,3R)-3-(7-(4-(dimethylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

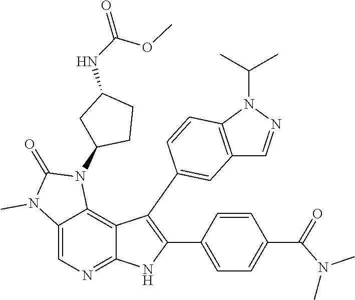

A procedure analogous to the one described in Example 8 was used to prepare the title compound. LCMS for C₃₅H₃₉N₈O₄ (M+H)⁺: m/z=635.3; Found: 635.2.

### Example 23. Methyl ((1R,3R)-3-(7-(4-(ethylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

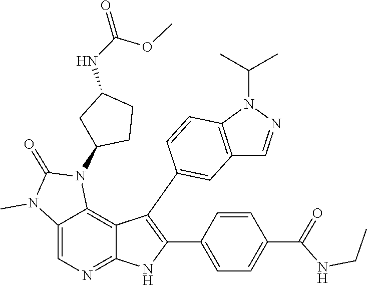 [SMILES: CCNC(=O)c1ccc(-c2[nH]c3ncc4c(c3c2-c2ccc3c(cnn3C(C)C)c2)n([C@@H]2CC[C@@H](NC(=O)OC)C2)c(=O)n4C)cc1]

A procedure analogous to the one described in Example 8 was used to prepare the title compound. LCMS for C₃₅H₃₉N₈O₄ (M+H)⁺: m/z=635.3; Found: 635.2.

### Example 24. Methyl ((1R,3R)-3-(7-(4-(4,4-difluoropiperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate

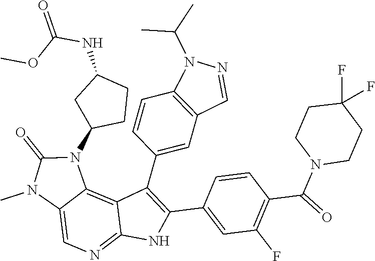 [SMILES: COC(=O)N[C@@H]1CC[C@@H](n2c(=O)n(C)c3cnc4[nH]c(-c5ccc(C(=O)N6CCC(F)(F)CC6)c(F)c5)c(-c5ccc6c(cnn6C(C)C)c5)c4c32)C1]

A procedure analogous to the one described in Example 8 was used to prepare the title compound. LCMS for C₃₈H₄₀F₃N₈O₄ (M+H)⁺: m/z=729.3; Found: 729.4.

### Example 25. Methyl ((1R,3R)-3-(7-(4-((bis(methyl-d3)amino)methyl)phenyl)-8-(2,2-dimethyl-2,3-dihydrobenzofuran-5-yl)-3-(methyl-d3)-2-oxo-3,6-dihydroimidazo[4,5-d]pyrrolo[2,3-b]pyridin-1(2H)-yl)cyclopentyl)carbamate

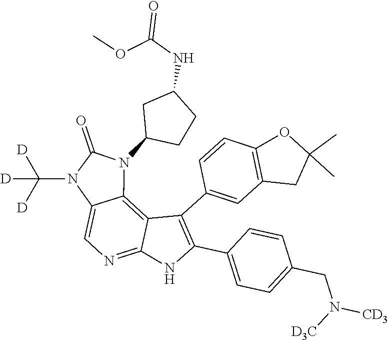 [SMILES: [2H]C([2H])([2H])N(Cc1ccc(-c2[nH]c3ncc4c(c3c2-c2ccc3c(c2)CC(C)(C)O3)n([C@@H]2CC[C@@H](NC(=O)OC)C2)c(=O)n4C([2H])([2H])[2H])cc1)C([2H])([2H])[2H]]

### Step 1. 3-Bromo-4-chloro-5-nitro-1-(phenylsulfonyl)-1H-pyrrolo[2,3-b]pyridine

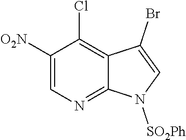

To a solution of 4-chloro-5-nitro-1-(phenylsulfonyl)-1H-pyrrolo[2,3-b]pyridine (5.0 g, 14.8 mmol; eNovation Chemicals, D572641) in dry dimethylformamide (30 mL), N-bromosuccinimide (5.0 g, 28.1 mmol) was added at room temperature. The resulting mixture was stirred for 60 minutes at 60° C. The yellow reaction mixture was cooled to 0° C. and kept at the same temperature for 6 hours. The resulting precipitate was collected using filtration followed by the wash with hexanes to give the desired product as a light yellow solid (5.35 g, 87.2%). LCMS for C₁₃H₈BrClN₃O₄S (M+H)⁺: m/z=415.9, 417.9; Found: 415.9, 417.9. ¹H NMR (600 MHz, DMSO) δ 9.06 (s, 1H), 8.54 (s, 1H), 8.19 (d, J=7.8 Hz, 2H), 7.80 (t, J=7.8 Hz, 1H), 7.68 (t, J=7.8 Hz, 2H).

### Step 2. tert-butyl ((1R,3R)-3-((3-bromo-5-nitro-1-(phenylsulfonyl)-1H-pyrrolo[2,3-b]pyridin-4-yl)amino)cyclopentyl)carbamate

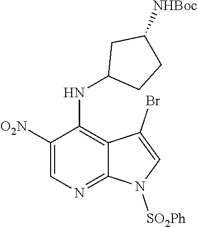

To a solution of 3-bromo-4-chloro-5-nitro-1-(phenylsulfonyl)-1H-pyrrolo[2,3-b]pyridine (25.17 g, 60.4 mmol) in 2-Propanol (302 mL) at 25° C. was sequentially added triethylamine (12.63 mL, 91 mmol) and tert-butyl ((1R,3R)-3-aminocyclopentyl) carbamate (15.12 g, 76 mmol). The reaction mixture was then heated to 100° C. and was allowed to stir for 15 min before checking progress by LC-MS. Upon completion, the solvent was removed in vacuo (azeotrope 2 times with toluene). The crude product (35.1 g, calculated as 60.5 mmol) was used directly in the next step. LC-MS calculated for C₂₃H₂₇BrN₅O₆S (M+H)⁺: m/z=580.09, 582.08; found 580.10, 582.10.

### Step 3. tert-butyl ((1R,3R)-3-((5-amino-3-bromo-1-(phenylsulfonyl)-1H-pyrrolo[2,3-b]pyridin-4-yl)amino)cyclopentyl)carbamate

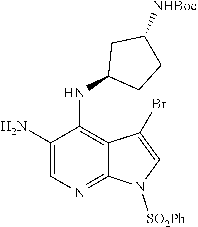

To the crude tert-butyl ((1R,3R)-3-((3-bromo-5-nitro-1-(phenylsulfonyl)-1H-pyrrolo[2,3-b]pyridin-4-yl)amino)cyclopentyl)carbamate directly from the last step (35.1 g, calculated as 60.5 mmol) was sequentially added THF (302 mL), Ethanol (101 mL), iron powder (16.87 g, 302 mmol), and HCl (36.2 mL, 36.2 mmol). The reaction mixture was then heated to 60° C. Reaction was monitored by LC-MS. Upon completion, the reaction was diluted with acetonitrile (50 mL) and stirred for 1 hour. The mixture was filtered through Celite, washed with DCM, and quenched further with K₂CO₃. The mixture was extracted with DCM, and the organic layer was concentrated to give the crude product as a gray amorphous solid (33.3 g calculated as 60.5 mmol), which was used in the next step without further purification. LC-MS calculated for C₂₃H₂₉BrN₅O₄S (M+H)⁺: m/z=550.11, 552.11; found 550.10, 552.10.

### Step 4. tert-butyl ((1R,3R)-3-(8-bromo-2-oxo-6-(phenylsulfonyl)-3,6-dihydroimidazo[4,5-d]pyrrolo[2,3-b]pyridin-1(2H)-yl)cyclopentyl)carbamate

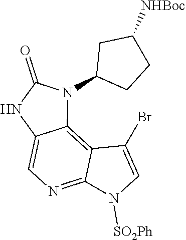

To a solution of crude tert-butyl ((1R,3R)-3-((5-amino-3-bromo-1-(phenylsulfonyl)-1H-pyrrolo[2,3-b]pyridin-4-yl)amino)cyclopentyl)carbamate directly from the previous step (33.3 g, calculated as 60.5 mmol) in THF (121 mL) was added carbonyldiimidazole (29.5 g, 182 mmol). The mixture was heated at 60° C. for 2 hours and cooled to 0° C. Water (10 mL) was added dropwise, with gas evolution noted initially. The reaction was warmed to room temperature and stirred for additional 30 minutes, before being diluted with 100 mL water and 100 mL EtOAc. The layers were separated, and the EtOAc extract was washed with 50 ml of water, 30 mL brine, dried, and filtered. The dried organic layer was concentrated to about 100 mL and left refrigerated overnight to crash out most of the product. The solid was filtered and washed with minimal amount of EtOAc to give 25.5 g product as a white amorphous solid (44.3 mmol 73.3% yield over 3 steps). LC-MS calculated for C₂₄H₂₇BrN₅O₅S (M+H)⁺: m/z=576.09, 578.09; found 576.10, 578.10.

### Step 5. tert-butyl ((1R,3R)-3-(8-bromo-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate

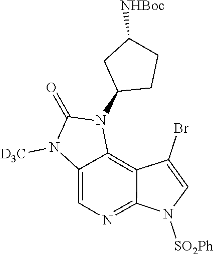

To a solution of tert-butyl ((1R,3R)-3-(8-bromo-2-oxo-6-(phenylsulfonyl)-3,6-dihydroimidazo[4,5-d]pyrrolo[2,3-b]pyridin-1(2H)-yl)cyclopentyl)carbamate (20.0 g, 34.7 mmol) in DMF (173 ml) at 40° C. was added iodomethane-d3 (3.3 ml, 52.0 mmol) and K₂CO₃ (6.2 g, 45.1 mmol). The reaction was allowed to stir at this temperature for 1 hour before monitoring by LC-MS. Upon completion, the reaction contents were poured into 400 mL of water, allowing for product to crash out, which was then filtered and washed with hexanes. The solid was redissolved in DCM and washed with brine. The organic layer was dried with MgSO₄ and concentrated to give a solid, which was then dissolved in 1:1 DCM/EtOAc (200 mL in total). The solution was then concentrated to crash out the product as a white solid, which was filtered and dried under vacuum. The collected mother liquor was concentrated and purified on silica gel (120 g, 0-100% EtOAc in DCM). 18.5 g product was collected as a white amorphous solid (18.5 g, 31.1 mmol, 89.8% yield). LC-MS calculated for C₂₅H₂₆D₃BrN₅O₅S (M+H)⁺: m/z=593.13, 595.12; found 593.10, 595.10.

### Step 6. methyl ((1R,3R)-3-(8-bromo-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate

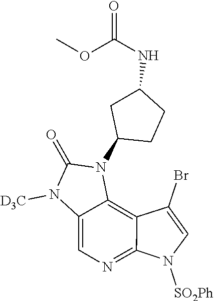

To a solution of tert-butyl ((1R,3R)-3-(8-bromo-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate (6.0 g, 10.1 mmol) in DCM (72 ml) was added trifluoroacetic acid (18.0 ml). The reaction mixture was then stirred at 25° C. for 1 hour. When LC-MS showed completion of the reaction, the mixture was concentrated directly and then azeotropically with toluene three times. The crude material was dissolved in DCM (90 ml) at 0° C., N,N-diisopropylethylamine (35.3 ml, 202 mmol) was added. The reaction was stirred at 0° C. for 5 minutes before the addition of methyl chloroformate (15.66 ml, 202 mmol). The reaction progress was then monitored by LCMS. Upon completion, the reaction was quenched by water and extracted with DCM. The organic layer was dried with MgSO₄ and concentrated. The crude residue was purified on silica gel (120 g, 0-100% EtOAc in DCM, then 0-20% methanol in DCM) to give 5.56 g product as a yellow to white amouphous solid (10.1 mmol, 99.7% yield). LC-MS calculated for C₂₅H₂₆D₃BrN₅O₅S (M+H)⁺: m/z=551.08, 553.08; found 551.10, 553.10.

### Step 7. methyl ((1R,3R)-3-(8-(2,2-dimethyl-2,3-dihydrobenzofuran-5-yl)-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate

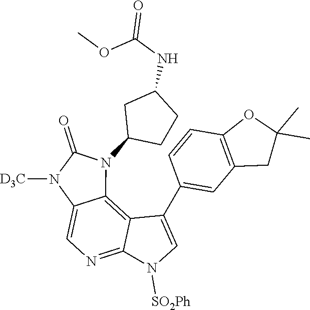

To a solution of methyl ((1R,3R)-3-(8-bromo-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate (1.65 g, 3.00 mmol) in 1,4-dioxane (12.00 mL) and water (3.00 mL) was added 2-(2,2-dimethyl-2,3-dihydrobenzofuran-5-yl)-4,4,5,5-tetramethyl-1,3,2-dioxaborolane (1.65 g, 6.00 mmol), K₂CO₃ (1.24 g, 9.00 mmol), and PdCl₂ (dppf)-CH₂Cl₂ adduct (0.74 g, 0.900 mmol). The resulting solution was then degassed with N₂ for 5 minutes before heating to 100° C. After 1 hour, LC-MS indicated complete conversion of starting materials. The reaction mixture was cooled to room temperature and partitioned between 50 mL EtOAc and 50 mL water. The aqueous layer was extracted with additional EtOAc, and the combined organic layer was washed with brine, dried with MgSO₄, and concentrated. The residue was purified on silica gel (40 g, 0-100% EtOAc in hexanes) to give 1.58 g product as a yellow to white amorphous solid (2.55 mmol, 85.0% yield). LC-MS calculated for C₃₂H₃₁D₃N₅O₆S (M+H)⁺: m/z=619.24; found 619.32.

### Step 8. methyl ((1R,3R)-3-(7-bromo-8-(2,2-dimethyl-2,3-dihydrobenzofuran-5-yl)-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate

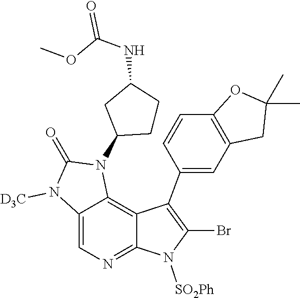

To a solution of methyl ((1R,3R)-3-(8-(2,2-dimethyl-2,3-dihydrobenzofuran-5-yl)-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate (618 mg, 0.99 mmol) in THF (10 mL) at −78° C. was added LDA (2 M in THF/heptane/ethylbenzen, Sigma-Aldrich, 1.398 mL, 2.80 mmol). The mixture was stirred for 30 minutes at −78° C. before introducing a solution of 1,2-dibromotetrachloroethane (342 mg, 1.05 mmol) in THF (1 mL). The mixture was stirred for additional 10 minutes at −78° C. before warming up to room temperature. LC-MS showed complete transformation of the starting material after warming up. The reaction was then quenched with 1 M HCl (2.8 mL) and partitioned between 20 mL brine and 20 mL EtOAc. The aqueous layer was extracted with additional EtOAc, and the combined organic layer was dried with MgSO₄ and concentrated. The residue was purified on silica gel (12 g, 0-100% EtOAc in hexanes) to give 560 mg product as a white amorphous solid (0.80 mmol, 81.0% yield). LC-MS calculated for C₃₂H₃₀D₃BrN₅O₆S (M+H)⁺: m/z=697.15, 699.15; found 697.28, 699.28.

### Step 9. Methyl ((1R,3R)-3-(7-(4-((bis(methyl-d3)amino)methyl)phenyl)-8-(2,2-dimethyl-2,3-dihydrobenzofuran-5-yl)-3-(methyl-d3)-2-oxo-3,6-dihydroimidazo[4,5-d]pyrrolo[2,3-b]pyridin-1(2H)-yl)cyclopentyl)carbamate

To a solution of methyl ((1R,3R)-3-(7-bromo-8-(2,2-dimethyl-2,3-dihydrobenzofuran-5-yl)-3-(methyl-d3)-2-oxo-6-(phenylsulfonyl)-3,6-dihydroimidazo[4,5-d]pyrrolo[2,3-b]pyridin-1(2H)-yl)cyclopentyl)carbamate (200 mg, 0.287 mmol) in Dioxane (4 mL) was sequentially added water (1 mL), Cs₂CO₃ (280 mg, 0.860 mmol), tetrakis(triphenylphosphine)palladium(0) (116 mg, 0.100 mmol), and N-(methyl-d3)—N-(4-(4,4,5,5-tetramethyl-1,3,2-dioxaborolan-2-yl)benzyl) methanamine-d3 (460 mg, 1.720 mmol, prepared by 1:1 mol ratio mixture of 2-(4-(bromomethyl)phenyl)-4,4,5,5-tetramethyl-1,3,2-dioxaborolane and d6-dimethyl amine in 3 mL THF and 200 mg Cs₂CO₃, followed by 10 minute stirring, filtration and concentration). The reaction mixture was degassed with N₂ and placed in a preheated pie block at 100° C. The reaction was allowed to stir for 30 min before checking progress by LC-MS. Upon completion, the reaction was cooled to room temperature, MeOH (5 mL) and aqueous NaOH solution (50% w/w, 0.5 mL) were added. The reaction was then stirred at 60° C. for 15 min before checking progress by LCMS. Upon completion, the reaction was diluted with MeCN, filtered, and purified by preparative HPLC (pH=2, then pH=10) to give 120 mg product as a white amorphous solid (120.5 mg, 0.195 mmol, 68.0% yield). LC-MS calculated for C₃₅H₃₂D₉N₆O₄ (M+H)⁺: m/z=618.38; found 618.50. ¹H NMR (600 MHz, DMSO) δ 12.38 (s, 1H), 9.86 (s, 1H), 8.22 (s, 1H), 7.54-7.36 (m, 4H), 7.21 (d, J=15.4 Hz, 1H), 7.16-7.05 (m, 1H), 6.93 (d, J=6.6 Hz, 1H), 6.86-6.76 (m, 1H), 4.22 (d, J=4.6 Hz, 2H), 4.17-3.96 (m, 2H), 3.49 (s, 3H), 3.22-3.01 (m, 1H), 2.97 (t, J=16.5 Hz, 1H), 2.15-1.82 (m, 3H), 1.46 (t, J=14.6 Hz, 6H), 1.30 (q, J=11.6 Hz, 1H), 0.79 (qd, J=11.8, 7.1 Hz, 1H).

### Examples 26-30

Examples 26-30 in Table 2 were prepared according to the procedures described in Example 25, using appropriately substituted starting materials.

TABLE 2

| Ex. | | | LCMS | |
|---|---|---|---|---|
| No. | Name | Structure | [M + H]⁺ | ¹H NMR |
| 26 | Methyl ((1R,3R)-3- (7-(4- ((dimethylamino)meth- yl)phenyl)-8-(1- isopropyl-1H- indazol-5-yl)-3- (methyl-d3)-2-oxo- 3,6- dihydroimidazo[4,5- d]pyrrolo[2,3- b]pyridin-1(2H)- yl)cyclopentyl)carba- mate | 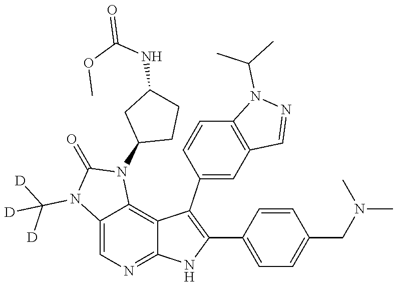 [SMILES: [2H]C([2H])([2H])n1c(=O)n([C@@H]2CC[C@@H](NC(=O)OC)C2)c2c3c(-c4ccc5c(cnn5C(C)C)c4)c(-c4ccc(CN(C)C)cc4)[nH]c3ncc21] | 624.3 | ¹H NMR (600 MHz, DMSO-d₆) 12.30 (s, 1H), 9.57 (s, 1H), 8.21 (s, 1H), 8.09 (s, 0.4H), 8.06 (s, 0.6H), 7.86-7.76 (m, 2H), 7.54- 7.40 (m, 3H), 7.37-7.32 (m, 2H), 6.67 (s, 0.6 H), 6.23 (s, 0.4H), 5.07 (m, 1H), 4.18 (s, 2H), 3.88 (m, 1H), 3.70 (m, 1H), 3.48 (s, 1.8 H), |
| | | | | 3.45 (s, 1.2 H), |
| | | | | 2.70 (s, 6H), |
| | | | | 2.03-1.93 (m, |
| | | | | 2H), 1.82-1.66 |
| | | | | (m, 1H), 1.61- |
| | | | | 1.45 (m, 7H), |
| | | | | 1.34 (m, 0.4H), |
| | | | | 1.21 (m, 0.6H), |
| | | | | 1.03 (m, 0.6 H), |
| | | | | 0.91 (m, 0.4H) |
| 27 | Methyl ((1R,3R)-3- (7-(4-((bis(methyl- d3)amino)methyl)phe- nyl)-8-(4-(2- methoxyethoxy)phe- nyl)-3-(methyl-d3)- 2-oxo-3,6- dihydroimidazo[4,5- d]pyrrolo[2,3- b]pyridin-1(2H)- yl)cyclopentyl)carba- mate | 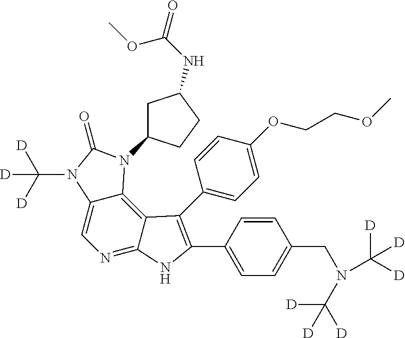 [SMILES: [2H]C([2H])([2H])N(Cc1ccc(-c2[nH]c3ncc4c(c3c2-c2ccc(OCCOC)cc2)n([C@@H]2CC[C@@H](NC(=O)OC)C2)c(=O)n4C([2H])([2H])[2H])cc1)C([2H])([2H])[2H]] | 622.4 | ¹H NMR (600 MHz, DMSO-d₆) 12.32 (s, 1H), 9.66 (s, 1H), 8.22 (s, 1H), 7.46 (d, J = 8.0 Hz, 2H), 7.40 (d, J = 8.0 Hz, 2H), 7.36 (d, J = 7.8 Hz, 1H), 7.32 (d, J = 7.8 Hz, 1H), 7.10 (d, J = 6.8 Hz, 1H), 7.06 (d, J = 6.8 Hz, 1H), 6.81 (d, J = 6.1 Hz, 1H), 4.23-4.14 (m, 4H), 4.06 (m, 1H), 3.87 (m, 1H), 3.72 (m, 2H), 3.51 (s, 3H), 3.35 (s, 3H), 2.07 |
| | | | | (m, 1H), 1.95 (m, |
| | | | | 1H), 1.87 (m, |
| | | | | 1H), 1.43 (m, |
| | | | | 1H), 1.29 (m, |
| | | | | 1H), 0.75 (m, |
| | | | | 1H) |
| 28 | Methyl ((1R,3R)-3- (7-(4- ((dimethylamino)meth- yl)phenyl)-8-(4- fluorophenyl)-3- methyl-2-oxo-3,6- dihydroimidazo[4,5- d]pyrrolo[2,3- b]pyridin-1(2H)- yl)cyclopentyl)carba- mate | 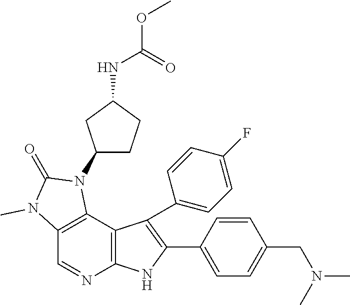 | 557.3 | ¹H NMR (600 MHz, DMSO-d₆) δ 12.15 (s, 1H), 8.16 (s, 1H), 7.44 (d, J = 7.8 Hz, 2H), 7.28 (m, 4H), 7.17 (d, J = 8.1 Hz, 2H), 6.93 (d, J = 6.9 Hz, 1H), 4.08 (m, 1H), 3.83 (m, 1H), 3.51 (s, 3H), 3.38 (s, 3H), 3.32 (s, 2H), 2.11 (s, 6H), 2.06 (m, 1H), 1.91 (m, 2H), 1.45 (m, 1H), 1.27 (q, J = 11.5 Hz, 1H), |
| | | | | 0.80 (m, 1H). |
| 29 | Methyl ((1R,3R)-3- (7-(4-((bis(methyl- d3)amino)methyl)phe- nyl)-8-(1,1- dimethyl-1,3- dihydroisobenzofuran- 5-yl)-3-(methyl- d3)-2-oxo-3,6- dihydroimidazo[4,5- d]pyrrolo[2,3- b]pyridin-1(2H)- yl)cyclopentyl)carba- mate | 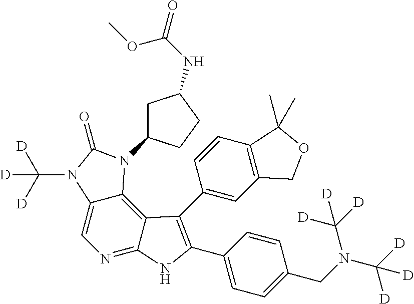 [SMILES: [2H]C([2H])([2H])N(Cc1ccc(-c2[nH]c3ncc4c(c3c2-c2ccc3c(c2)COC3(C)C)n([C@@H]2CC[C@@H](NC(=O)OC)C2)c(=O)n4C([2H])([2H])[2H])cc1)C([2H])([2H])[2H]] | 618.4 | ¹H NMR (600 MHz, DMSO) δ 12.33 (s, 1H), 9.60 (s, 1H), 8.21 (s, 1H), 7.44-7.30 (m, 7H), 7.00 (s, 0.5 H), 6.83 (s, 0.5H), 5.16 (m, 1H), 4.96 (m, 1H), 4.21 (d, J = 4.8 Hz, 2H), 4.08 (m, 1H), 3.91 (m, 1H), 3.50 (m, 3H), 2.10-1.81 (m, 3H), 1.51 (m, 6H), 1.39 (m, 1H), 1.23 (m, 1H), 0.70 (m, 1H) |
| 30 | methyl ((1R,3R)-3- (8-(1,1-dimethyl- 1,3- dihydroisobenzofuran- 5-yl)-7-(4- (dimethylcarbamoyl) phenyl)-3-(methyl- d3)-2-oxo-3,6- dihydroimidazo[4,5- d]pyrrolo[2,3- b]pyridin-1(2H)- yl)cyclopentyl)carba- mate | 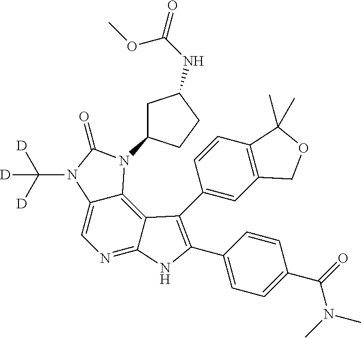 [SMILES: [2H]C([2H])([2H])n1c(=O)n([C@@H]2CC[C@@H](NC(=O)OC)C2)c2c3c(-c4ccc5c(c4)COC5(C)C)c(-c4ccc(C(=O)N(C)C)cc4)[nH]c3ncc21] | 626.3 | ¹H NMR (600 MHz, DMSO-d₆) δ 12.34 (s, 1H), 8.21 (s, 1H), 7.46- 6.70 (m, 8H), 5.20-4.86 (m, 2H), 4.13-3.89 (m, 3H), 3.48 (d, J = 4.3 Hz, 3H), 3.01-2.83 (m, 5H), 2.10-1.80 (m, 3H), 1.49 (d, J = 5.0 Hz, 6H), 1.39 (dt, J = 11.2, 7.6 Hz, 1H), 1.22 (q, J = 11.5 Hz, 1H), 0.69 (d, J = 11.4 Hz, 1H). |

### Example A. JAK2 LanthaScreen JH1 Binding Assay

JAK2 JH1 binding assay utilizes catalytic domain (JH1, amino acids 826-1132) of human JAK2 expressed as N-terminal FLAG-tagged, biotinylated protein in a baculovirus expression system (Carna Biosciences, Product #08-445-20N). The assay was conducted in black 384-well polystyrene plates in a final reaction volume of 20 μL. JAK2 JH1 (1.5 nM) was incubated with compounds (100 nL serially diluted in DMSO) in the presence of 50 nM fluorescent JAK2-JH1 tracer and 0.5 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 2 hours at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example B. JAK2 LanthaScreen JH2-V617F Binding Assay

JAK2 JH2-V617F binding assay utilizes pseudo-kinase domain (JH2, amino-acids 536-812 with 3 surface mutations W659A, W777A, F794H) of human V617F mutant JAK2 expressed as C-terminal His-Avi-tagged, biotinylated protein in a baculovirus expression system (BPS Bioscience, Catalog #79498). The assay was conducted in black 384-well polystyrene plates in a final reaction volume of 20 μL. JAK2 JH2-V617F (0.26 nM) was incubated with compounds (100 nL serially diluted in DMSO) in the presence of 50 nM Fluorescent JAK2-JH2 Tracer (MedChem Express Catalog #HY-102055) and 0.25 nM Streptavidin-Tb cryptate (Cisbio Part #610SATLB) in assay buffer (50 mM Tris, pH=7.5, 10 mM MgCl₂, 0.01% Brij-35, 0.1% BSA, 1 mM EGTA, 5% Glycerol and 5 mM DTT). Non-specific binding was accessed in the presence of 2 mM ATP. After incubation for 1 hour at 25° C., LanthaScreen signals were read on a PHERAstar FS plate reader (BMG LABTECH). Data was analyzed with IDBS XLfit and GraphPad Prism 5.0 software using a four parameter dose response curve to determine IC50 for each compound.

### Example C. FLT3 Enzymatic Assay

The kinase assays were carried out at room temperature in assay buffer (HEPES 50 mM, pH 7.0, NaN₃ 0.02%, BSA 0.01%, Orthovanadate 0.1 mM, DTT 1 mM, MgCl₂ 10 mM) in a final volume of 10 μL. Testing compounds were prepared by serial dilution in DMSO and transferred to the plate wells by ECHO liquid handler (Labcyte) with 0.5% DMSO in the final assay. The FLT3/TK Substrate-biotin mixture is prepared in the assay buffer with 1000 nM TK Substrate-biotin and SEB reagent 125 nM. 5 μL mixture was added to polystyrene 384-well small volume black plate (Greiner Bio-One). Reactions were initiated by the addition of 5 μL ATP in assay buffer. The final 10 μL kinase reaction consists of 0.011 nM FLT3, 1 mM ATP, 500 nM TK Substrate-biotin and SEB reagent 62.5 nM in assay buffer. Reactions were incubated for 90 min and terminated by addition of 10 μL of detection reagent containing 125 nM Streptavidin-XL665, TK Antibody-Cryptate in HTRF® Detection buffer (HEPES 50 mM, pH 7.0, BSA 0.1%, KF 0.8 M, EDTA 20 mM). The plates were then sealed and centrifuged at 1800 rpm for 2 minutes. After 60 minutes incubation at room temperature, the product activity was determined by measuring the fluorescence at 620 nm and 665 nm on Pherastar microplate reader (BMG Labtech). A ratio is calculated (665/620 nm) for each well. Wells with DMSO only were served as the positive controls and wells containing no ATP were used as negative controls. IC₅₀ determination was performed by fitting the curve of percent control activity versus the log of the compound concentration using the GraphPad Prism 7.0 software.

### Example D. TRKA Enzymatic Assay

The kinase assays were carried out at 24° C. temperature in assay buffer (HEPES 50 mM, pH 7.0, NaN₃ 0.02%, BSA 0.01%, Orthovanadate 0.1 mM, DTT 1 mM, MgCl₂ 10 mM) in a final volume of 10 μL. Testing compounds were prepared by serial dilution in DMSO and transferred to the plate wells by ECHO liquid handler (Labcyte) with 0.5% DMSO in the final assay. The TRKA/TK Substrate-biotin mixture is prepared in the assay buffer with 1000 nM TK Substrate-biotin and SEB reagent 125 nM. 5 μL mixture was added to polystyrene 384-well small volume black plate (Greiner Bio-One). Reactions were initiated by the addition of 5 μL ATP in assay buffer. The final 10 μL kinase reaction consisted of 0.58 nM TRKA, 1 mM ATP, 500 nM TK Substrate-biotin and SEB reagent 62.5 nM in assay buffer. Reactions were incubated for 90 min and terminated by addition of 10 μL of detection reagent containing 125 nM Streptavidin-XL665, TK Antibody-Cryptate in HTRF® Detection buffer (HEPES 50 mM, pH 7.0, BSA 0.1%, KF 0.8 M, EDTA 20 mM). After 60 minutes incubation at room temperature, the product activity was determined by measuring the fluorescence at 620 nm and 665 nm on Pherastar microplate reader (BMG Labtech). A ratio was calculated (665/620 nm) for each well. Wells with DMSO only served as the positive controls and wells containing no ATP were used as negative controls. IC₅₀ determination was performed by fitting the curve of percent control activity versus the log of the compound concentration using the Gene data.

### Example D2. Time Dependent Inhibition (TDI) of CYP Protocol

Inhibition of P450 enzymes can be classified as either reversible or time-dependent inhibition (TDI). This in vitro assay is designed to detect an increase in CYP inhibition as a result of biotransformation and/or bioactivation (TDI). Potential mechanisms for this include the formation of a tight-binding, quasi-irreversible inhibitory metabolite complex or the inactivation of P450 enzymes by covalent adduct formation of metabolites. While this experiment employs a 10-fold dilution to diminish metabolite concentrations and therefore effects of reversible inhibition, it is possible (but not common) that a metabolite that is an extremely potent CYP inhibitor could result in a positive result.

The results are from a cocktail of CYP specific probe substrates at 4 times their Km concentrations for CYP2C9 (diclofenac), CYP2C19 (S-mephenytoin), CYP2D6 (bufuralol) and CYP3A4 (midazolam) using human liver microsomes (HLM). The HLMs were pre-incubated with test compound(s) at a concentration 10 μM for 30 min in the presence (+N) or absence (−N) of a NADPH regenerating system, diluted 10-fold, and incubated for 8 min in the presence of the substrate cocktail with the addition of a fresh aliquot of NADPH regenerating system. A calibration curve of metabolite standards was used to quantitatively measure the enzyme activity using LC-MS/MS. In addition, incubations with known time dependent inhibitors, tienilic aicd (CYP2C9), ticlopidine (CYP2C19), paroxetine (CYP2D6), and troleandomycin (CYP3A4), used as positive controls were pre-incubated 30 min with or without a NADPH regenerating system.

The percentage of control CYP2C9, CYP2C19, CYP2D6, and CYP3A4 activity remaining following pre-incubation of the compounds with NADPH is corrected for the corresponding control vehicle activity and then calculated based on 0 minutes as 100%. A linear regression plot of the natural log of % activity remaining versus time for each isozyme is used to calculate the slope. The −slope is equal to the rate of enzyme loss, or the K_{obs}. Comparative Examples A and B exhibit K_{obs} around 0.04 indicating a high potential for time dependent inhibition of CYPs corresponding to about 30% CYP function remaining. Examples 25-28 have Kobs <0.005 indicating a low potential for time dependent inhibition of CYPs corresponding to >90% CYP function remaining. Examples 29-30 have Kobs from 0.005-0.02 indicating a medium to low potential for time dependent inhibition of CYPs corresponding to 70-90% CYP function remaining.

### Example D3. FaSSIF Solubility Assay

The FaSSIF (Fasted State Simulated Intestinal Fluid) solubility assay can predict how an oral drug is likely to be dissolved and potentially absorbed in the upper intestine after drinking a glass of water. FaSSIF solubility assay utilizes biorelevant intestinal media (Biorelevant, Part #FFF01) comprised of physiological surfactants, bile salts, and phospholipids that are present in the gastrointestinal fluids it mimics. FaSSIF (pH=6.5) has the average pH of a fasted intestinal fluid with similar osmolarity. The FaSSIF buffer was prepared by adding 20 mL of phosphate buffer to FaSSIF Powder. The buffer was then added to the dry compound of interest at a concentration of 1 mg/mL and then incubated at 25° C. for 24 hours in a Rototherm Plus (Part #H2024). After incubation, the Eppendorf tubes were centrifuged and run on an Agilent 1260 LC-MS. The LC-MS method is a 5 min gradient method of 2% to 95% acetonitrile with water (0.05% TFA) using a Sunfire C18, 5 um 2.1×50 mm (Part #186002539) column. A dry reference compound of interest was diluted to a known 1 mg/mL concentration in 1:1 of water and acetonitrile. Step-wise serial dilutions were performed and run on an Agilent 1260 LC-MS using the same conditions to make a standard curve. The area under the curve is reported for the compound of interest in FaSSIF buffer and the reference standards. Calculations were performed and the predicted solubility, μg/mL, was reported for compounds of the examples. The FaSSIF solubility of comparative Examples A, D and F was less than 20 μg/mL which indicated poor solubility while Examples 25-29 exhibited FaSSIF solubility >100 μg/mL, which indicated good solubility.

The compounds of the disclosure and comparative examples were tested in one or more of the assays described in Examples A-D₂, and the resulting data are shown in Tables A1-A2. Selectivity against tyrosine kinases such as FLT3 and TRKA are desirable. Clinical studies have shown that inhibitors of TRKA and FLT3 are myelosuppressive, most likely due to the roles that these kinases play in the hematopoietic system. Both TRKA and FLT3 are expressed predominantly in hematopoietic stem and progenitor cells. TRKA may play a roll in normal hematopoiesis. Likewise mouse genetic studies have shown that FLT3 knockout mice have deficiencies in abilities to repopulate B and T cells. Combined knockout of TRKA and FLT3 results in lethality in mice.

TABLE A

| | JH2 | | | | | |
|---|---|---|---|---|---|---|
| | V617F | JH1 | | | | |
| | Bind | Bind | FLT3 | TRKA | Selectivity | Selectivity |
| | IC₅₀ | IC₅₀ | IC₅₀ | IC₅₀ | FLT3/JH2 | TRKA/JH2 |
| Example No. | (nM) | (nM) | (nM) | (nM) | V617F | V617F |
| Comparative | + | +++++ | ††† | †††† | * | ** |
| Example A | | | | | | |
| Comparative | + | +++++ | ††† | ††††† | * | ** |
| Example B | | | | | | |
| Comparative | + | ++ | † | † | * | * |
| Example C | | | | | | |
| Comparative | + | +++ | NT | NT | NT | NT |
| Example D | | | | | | |
| Comparative | + | +++++ | ††† | ††† | * | ** |
| Example E | | | | | | |
| Comparative | + | ++++ | †† | †† | * | * |
| Example F | | | | | | |
| Comparative | +++++ | +++ | NT | NT | NA | NA |
| Example G | | | | | | |
| Comparative | + | +++ | †† | †† | * | * |
| Example H | | | | | | |
| Comparative | + | +++ | † | † | * | * |
| Example I | | | | | | |
| 1 | + | +++++ | NT | NT | NT | NT |
| 2 | + | +++++ | NT | NT | NT | NT |
| 3 | + | +++++ | NT | NT | NT | NT |
| 4 | + | +++++ | NT | NT | NT | NT |
| 5 | + | +++++ | †††† | ††† | ***** | ** |
| 6 | + | +++++ | †††† | †††† | ***** | ***** |
| 7 | + | +++++ | ††††† | ††††† | ***** | ***** |
| 8 | + | +++++ | ††††† | ††††† | ****** | ****** |
| 9 | + | +++++ | ††††† | ††††† | ****** | ****** |
| 10 | + | +++++ | ††††† | ††††† | ****** | ****** |
| 11 | + | +++++ | ††††† | ††††† | ****** | ****** |
| 12 | + | +++++ | ††††† | ††††† | ****** | ****** |
| 13 | + | +++++ | ††††† | ††††† | ****** | ***** |
| 14 | + | +++++ | ††††† | ††††† | ****** | ****** |
| 15 | + | +++++ | †††† | ††††† | ****** | ****** |
| 16 | + | +++++ | †††† | †††† | ****** | ****** |
| 17 | + | +++++ | NT | NT | NT | NT |
| 18 | + | +++++ | ††† | †††† | **** | ***** |
| 19 | + | +++++ | ††† | †††† | **** | ****** |
| 20 | + | +++++ | ††† | ††† | ***** | ***** |
| 21 | + | +++++ | ††† | †††† | ***** | ****** |
| 22 | + | +++++ | ††† | ††† | ***** | ***** |
| 23 | + | +++++ | ††† | †††† | **** | ***** |
| 24 | + | +++++ | ††††† | ††††† | ****** | ****** |
| 25 | + | +++++ | ††† | ††† | **** | **** |
| 26 | + | +++++ | ††† | †††† | **** | ***** |
| 27 | + | +++++ | ††† | †††† | *** | ***** |
| 28 | + | +++++ | ††† | ††††† | ** | **** |
| 29 | + | +++++ | ††† | †††† | **** | ****** |
| 30 | + | +++++ | ††† | ††† | ***** | ***** |

NT refers to “not tested”
+ refers to IC₅₀ of ≤10 nM
++ refers to IC₅₀ of >10 nM to ≤100 nM
+++ refers to IC₅₀ of >100 nM to ≤500 nM
++++ refers to IC₅₀ of >500 nM to ≤1000 nM
+++++ refers to IC₅₀ of >1000 nM
† refers to IC₅₀ of ≤25 nM
†† refers to IC₅₀ of >25 nM to ≤250 nM
††† refers to IC₅₀ of >250 nM to ≤1250 nM
†††† refers to IC₅₀ of >1250 nM to ≤2500 nM
††††† refers to IC₅₀ of >2500 nM
* refers to selectivity of ≤500-fold
** refers to selectivity of >500-fold to ≤1500-fold
*** refers to selectivity of >1500-fold to ≤1750-fold
**** refers to selectivity of >1750-fold to ≤5000-fold
***** refers to selectivity of >5000-fold to ≤12500-fold
****** refers to selectivity of >12500-fold

TABLE A2

| | | Solubility |
|---|---|---|
| Example | | Fassif |
| No. | TDI 3A4 | (μg/mL) |
| Comparative | ⋄⋄⋄⋄ | ▴ |
| Example A | | |
| Comparative | ⋄⋄⋄⋄ | ▴ |
| Example B | | |
| Comparative | NT | NT |
| Example C | | |
| Comparative | NT | NT |
| Example D | | |
| Comparative | NT | ▴ |
| Example E | | |
| Comparative | NT | ▴ |
| Example F | | |
| Comparative | NT | NT |
| Example G | | |
| Comparative | NT | NT |
| Example H | | |
| Comparative | NT | NT |
| Example I | | |
| 1 | NT | NT |
| 2 | NT | NT |
| 3 | NT | NT |
| 4 | NT | NT |
| 5 | NT | NT |
| 6 | NT | NT |
| 7 | NT | NT |
| 8 | NT | NT |
| 9 | NT | NT |
| 10 | NT | NT |
| 11 | NT | NT |
| 12 | NT | NT |
| 13 | NT | NT |
| 14 | NT | NT |
| 15 | NT | NT |
| 16 | NT | NT |
| 17 | NT | NT |
| 18 | NT | NT |
| 19 | NT | NT |
| 20 | NT | NT |
| 21 | NT | NT |
| 22 | NT | NT |
| 23 | NT | NT |
| 24 | NT | NT |
| 25 | ⋄ | ▴▴ |
| 26 | ⋄ | ▴▴▴ |
| 27 | ⋄ | ▴▴▴ |
| 28 | ⋄ | ▴▴▴ |
| 29 | ⋄⋄ | ▴▴▴ |
| 30 | ⋄⋄ | NT |

⋄ refers to ≤0.005
⋄⋄ refers to >0.005 to ≤0.02
⋄⋄⋄ refers to >0.02 to <0.04
⋄⋄⋄⋄ refers to ≥0.04
▴ refers to ≤50
▴▴ refers to >50 to ≤500
▴▴▴ refers to >500

### Example E. STAT5 (Tyr694) Phosphorylation Cell Based Assay

SET-2 cells was purchased from DSMZ (Germany). RPMI1640 medium, Fetal Bovine Serum and 384-white sold flat-bottom small volume plate were purchased from Thermo Fisher Scientific (Waltham, MA). Phospho-STAT5 (Tyr694) HTRF kit was purchased from Perkin Elmer (Waltham, MA).

SET-2 cells were cultured in RPMI media with 20% FBS at 37° C. in humidified incubator supplied with 5% CO₂. On the day of assay, the cells were centrifuged to remove the culture media and resuspended with prewarmed RPMI with 10% FBS. Testing compounds were prepared by serial dilution in DMSO and 50 nL/well test compounds were transferred to the 384 white low volume cell culture plate (Greiner Bio-one) by ECHO liquid handler (Labcyte). The cells were then dispensed with Multidrop (Thermo Fisher, Waltham, MA) at 10 μL/well (7×10⁶ cells/mL) with 0.5% DMSO in the final assay. After the treated cells were incubated for 2 hours at 37° C./5% CO₂ incubator, 4 μL/well supplemented lysis buffer (100× blocking buffer diluted 25 fold in 4× lysis buffer, Perkin Elmer) were added and incubated at room temperature for 90 min on orbital shaker at 600 rpm. Phospho-STAT5 Cryptate antibody and Phospho-STAT5 d2 antibody (1:1 vol/vol) were premixed and diluted 20 fold with in the detection buffer (Perkin Elmer). 4 μL of the premixed antibody solution were added to each well followed with 24 hours incubation at room temperature on orbital shaker at 600 rpm. The product activity was determined by measuring the fluorescence at 620 nm and 665 nm on Pherastar microplate reader (BMG Labtech). A ratio is calculated (665/620) for each well. Wells with DMSO only served as the positive controls and wells containing high concentration of control compound were used as negative controls. IC₅₀ determination was performed by fitting the curve of percent control activity versus the log of the compound concentration using the GraphPad Prism 7.0 software.

### Example F. pSTAT5 SET-2 Whole Blood MSD Assay

SET-2 cells shipped from ABS, RPMI1640 medium, Fetal Bovine Serum from Gibco, Phospho-STAT5a,b Whole Cell Lysate Kit from Mesoscale, Retronectin (Recombinant Human Fibronectin Fragment) from TaKaRa, 96 well cell culture plate flat bottom from Corning, lysis buffer from Cell Signaling Technology, and sterile PBS from Gibco. Whole Blood from by BioIVT.

SET-2 cells were cultured in RPMI media with 20% FBS at 37° C. humidified incubator supplied with 5% CO₂. On the day of the assay, 40 uL per well of diluted retronectin working solution was added to sterile 96-well clear flat bottom tissue culture plates (1:200 dilution of retronectin in sterile PBS) and incubated at 37 C for 1 hour. After 1 hour, the retronectin working solution was aspirated and SET-2 cells were seeded at 100,000/well in RPMI+20% FBS. Cells were incubated overnight at 37° C. and 5% CO₂. The next day, the medium was removed and 40 μL of diluted compounds in serum-free RPMI and whole blood was added to the cells for a 2 hour incubation at 37° C. and 5% CO₂. The compound starting concentration is 20 μM and 2.5 fold serial diluted down to 0.21 nM. The whole blood and compound mixture was then aspirated off using the BlueCat plate washer and washed 1× with PBS. Cells were lysed by adding 40 μL of 3× complete lysis buffer and shaken at room temperature for 45-60 minutes (complete lysis buffer consists of Cell Signaling Technology lysis buffer diluted to 3× and supplemented with Mesoscale's protease inhibitor, phosphatase I inhibitor, and phosphatase II inhibitor). The pSTAT5 MSD plates were blocked by adding 150 μL of Blocker A solution per well to the MSD plates and incubated at room temperature with shaking at 400 rpm for one hour or longer. After blocking, plates were washed 3× with 300 μL/well of 1× Tris Wash Buffer. After lysis, added 25 μL per well of lysed sample was added to MSD plate and incubated with shaking at 4° C. wrapped in foil overnight. The next day, plates were washed again and 25 μL/well of diluted 1× Detection Antibody Solution, wrapped in foil, and incubated at room temperature with shaking at 400 rpm for 1 hour. Plates were then washed again and lastly, 150 μL/well of 1× Read Buffer T was added to all wells. Plates were analyzed on MSD Discovery within 5 minutes. Inhibition of pSTAT5 signaling was calculated against a control as inhibitory concentration for 50% inhibition (IC₅₀). Data analysis was performed in GraphPad Prism using a 4-parameter fit and data was reported as Average±SD.

### Example G. In Vitro Intrinsic Clearance Protocol

For in vitro metabolic stability experiments, test compounds were incubated with human liver microsomes at 37° C. The incubation mixture contained test compounds (1 μM), NADPH (2 mM), and human liver microsomes (0.5 mg protein/mL) in 100 mM phosphate buffer (pH 7.4). The mixture was pre-incubated for 2 min at 37° C. before the addition of NADPH. Reactions were commenced upon the addition of NADPH and quenched with ice-cold methanol at 0, 10, 20, and 30 min. Terminated incubation mixtures were analyzed using LC-MS/MS system. The analytical system consisted of a Shimadzu LC-30AD binary pump system and SIL-30AC autosampler (Shimadzu Scientific Instruments, Columbia, MD) coupled with a Sciex Triple Quad 6500+ mass spectrometer from Applied Biosystems (Foster City, CA). Chromatographic separation of test compounds and internal standard was achieved using a Hypersil Gold C18 column (50×2.1 mm, 5 μM, 175 Å) from ThermoFisher Scientific (Waltham, MA). Mobile phase A consisted of 0.1% formic acid in water, and mobile phase B consisted of 0.1% formic acid in acetonitrile. The total LC-MS/MS runtime was 2.75 minutes with a flow rate of 0.75 mL/min. Peak area integrations and peak area ratio calculations were performed using Analyst software (version 1.6.3) from Applied Biosystems.

The in vitro intrinsic clearance, CL_{int, in vitro}, was calculated from the t_{1/2} of test compound disappearance as CL_{int, in vitro}=(0.693/t_{1/2})×(1/Cₚᵣₒₜₑᵢₙ), where Cₚᵣₒₜₑᵢₙ is the protein concentration during the incubation, and t_{1/2} was determined by the slope (k) of the log-linear regression analysis of the concentration versus time profiles; thus, t_{1/2}=ln2/k. The CL_{int, in vitro} values were scaled to the in vivo values for human by using physiologically based scaling factors, hepatic microsomal protein concentrations (45 mg protein/g liver), and liver weights (21 g/kg body weight). The equation CLᵢₙₜ=CL_{int, in vitro}×(mg protein/g liver weight)×(g liver weight/kg body weight) was used. The in vivo hepatic clearance (CL_{H}) was then calculated by using CLᵢₙₜ and hepatic blood flow, Q (20 mL·min⁻¹·kg⁻¹ in humans) in the well-stirred liver model disregarding all binding from CL_{H}=(Q×CLᵢₙₜ)/(Q+CLᵢₙₜ). The hepatic extraction ratio was calculated as CL_{H} divided by Q.

### Example H. In Vivo Pharmacokinetics

Test compounds were administered to male Sprague Dawley rats or male and female Cynomolgus monkeys intravenously or via oral gavage. For intravenous (IV) dosing, test compounds were dosed at 1 mg/kg using a formulation of 10% dimethylacetamide (DMAC), 10% propylene glycol (PG) in acidified saline via IV bolus for rat or 10 min IV infusion for monkey. For oral (PO) dosing, test compounds were dosed at 3.0 mg/kg using 5% DMAC in 0.5% methylcellulose in citrate buffer (PH˜2.5). Blood samples were collected at predose and various time points up to 24 hours postdose. All blood samples were collected using EDTA as the anticoagulant and centrifuged to obtain plasma samples. The plasma concentrations of test compounds were determined by LC-MS/MS methods. The measured plasma concentrations were used to calculate PK parameters by standard noncompartmental methods using Phoenix® WinNonlin software program (version 8.0, Pharsight Corporation) or similar software. In rats and monkeys, cassette dosing of test compounds were conducted to obtain preliminary PK parameters. In vivo pharmacokinetic experiments with male beagle dogs may be performed under the conditions described above.

The compounds of the disclosure and comparative examples were tested in one or more of the assays described in Examples E-H, and the resulting data are shown in Table B.

TABLE B

| | SET2 | SET2 | | RAT PO | CYNO PO |
|---|---|---|---|---|---|
| Example | IC₅₀ | WB IC₅₀ | h-IntCL | AUC | AUC |
| No. | (nM) | (nM) | (L/h/kg) | (nM * h) | (nM * h) |
| Comparative | [symbol] [symbol] [symbol] | [symbol] [symbol] [symbol] | [symbol] [symbol] | [symbol] [symbol] [symbol] | [symbol] |
| Example A | | | | (mouse) | |
| Comparative | [symbol] [symbol] [symbol] | [symbol] [symbol] [symbol] | [symbol] | [symbol] [symbol] [symbol] | [symbol] |
| Example B | | | | | |
| Comparative | [symbol] | NT | [symbol] [symbol] [symbol] | NT | NT |
| Example C | | | | | |
| Comparative | [symbol] | NT | [symbol] [symbol] [symbol] | NT | NT |
| Example D | | | | | |
| Comparative | [symbol] [symbol] | [symbol] [symbol] [symbol] [symbol] | [symbol] [symbol] [symbol] | [symbol] [symbol] [symbol] | NT |
| Example E | | | | (mouse) | |
| Comparative | [symbol] [symbol] | NT | [symbol] [symbol] [symbol] | NT | NT |
| Example F | | | | | |
| Comparative | NT | NT | NT | NT | NT |
| Example G | | | | | |
| Comparative | [symbol] [symbol] | NT | [symbol] | NT | NT |
| Example H | | | | | |
| Comparative | [symbol] [symbol] | NT | [symbol] [symbol] [symbol] | NT | NT |
| Example I | | | | | |
| 1 | [symbol] [symbol] [symbol] | NT | NT | NT | NT |
| 2 | [symbol] [symbol] [symbol] | NT | NT | NT | NT |
| 3 | [symbol] [symbol] [symbol] | NT | NT | NT | NT |
| 4 | [symbol] [symbol] [symbol] | NT | NT | NT | NT |
| 5 | [symbol] [symbol] [symbol] | NT | [symbol] [symbol] [symbol] | NT | NT |
| 6 | [symbol] | [symbol] [symbol] | [symbol] [symbol] | NT | NT |
| 7 | [symbol] [symbol] [symbol] | NT | [symbol] [symbol] | NT | NT |
| 8 | [symbol] [symbol] | [symbol] [symbol] [symbol] | [symbol] [symbol] | NT | NT |
| 9 | [symbol] [symbol] | [symbol] [symbol] [symbol] | [symbol] | NT | NT |
| 10 | [symbol] [symbol] | [symbol] [symbol] [symbol] [symbol] | [symbol] [symbol] | NT | NT |
| 11 | [symbol] | [symbol] [symbol] [symbol] [symbol] | [symbol] | NT | NT |
| 12 | [symbol] | [symbol] [symbol] [symbol] [symbol] | [symbol] [symbol] | NT | NT |
| 13 | [symbol] [symbol] | [symbol] [symbol] [symbol] | [symbol] | NT | NT |
| 14 | [symbol] | [symbol] [symbol] [symbol] | [symbol] [symbol] [symbol] | NT | NT |
| 15 | [symbol] | [symbol] [symbol] [symbol] | [symbol] [symbol] [symbol] | NT | NT |
| 16 | [symbol] | [symbol] [symbol] | [symbol] [symbol] | NT | NT |
| 17 | [symbol] [symbol] [symbol] | NT | NT | NT | NT |
| 18 | [symbol] | NT | [symbol] [symbol] | NT | NT |
| 19 | [symbol] | NT | [symbol] [symbol] | NT | NT |
| 20 | [symbol] | NT | [symbol] [symbol] | NT | NT |
| 21 | [symbol] [symbol] | [symbol] [symbol] [symbol] [symbol] | [symbol] [symbol] | NT | NT |
| 22 | [symbol] | NT | [symbol] [symbol] | NT | NT |
| 23 | [symbol] | [symbol] [symbol] | [symbol] [symbol] | NT | NT |
| 24 | [symbol] [symbol] | NT | [symbol] [symbol] | NT | NT |
| 25 | [symbol] | [symbol] | [symbol] [symbol] | [symbol] [symbol] | [symbol] |
| | | | | | (1 mg/kg) |
| 26 | [symbol] | [symbol] | [symbol] [symbol] | [symbol] | [symbol] [symbol] |
| 27 | [symbol] | [symbol] | [symbol] [symbol] | [symbol] [symbol] | [symbol] [symbol] [symbol] |
| 28 | [symbol] [symbol] | [symbol] | [symbol] [symbol] | [symbol] [symbol] | NT |
| 29 | [symbol] | [symbol] | [symbol] [symbol] | [symbol] | NT |
| 30 | [symbol] | [symbol] | [symbol] [symbol] | [symbol] | NT |

NT refers to “not tested” [symbol]
refers to IC₅₀ of ≤300 nM [symbol] [symbol]
refers to IC₅₀ of >300 nM to ≤800 nM [symbol] [symbol] [symbol]
refers to IC₅₀ of >800 nM [symbol]
refers to IC₅₀ of ≤2500 nM [symbol] [symbol]
refers to IC₅₀ of >2500 nM to ≤5000 nM [symbol] [symbol] [symbol]
refers to IC₅₀ of >5000 nM to ≤7500 nM [symbol] [symbol] [symbol] [symbol]
refers to IC₅₀ of >7500 nM [symbol]
refers to h-IntCL of <0.7 L/h/kg [symbol] [symbol]
refers to h-IntCL of ≥0.7 L/h/kg to ≤1 L/h/kg [symbol] [symbol] [symbol]
refers to h-IntCL of >1 L/h/kg [symbol]
refers to AUC of ≤500 nM * h [symbol] [symbol]
refers to AUC of >500 nM * h to ≤1500 nM * h [symbol] [symbol] [symbol]
It refers to AUC of >1500 nM * h

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A compound of Formula I: 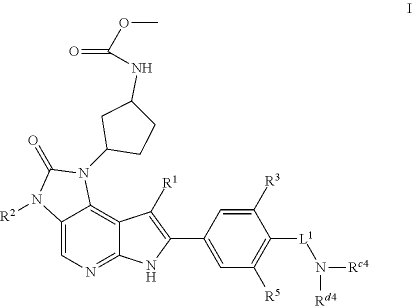
or a pharmaceutically acceptable salt thereof, wherein:
L¹ is C₁₋₃ alkylene or C(O);
R¹ is selected from phenyl, dihydrobenzofuranyl, dihydroisobenzofuranyl, and indazolyl, each of which is optionally substituted by 1, 2, or 3 substituents independently selected from halo, C₁₋₆ alkyl, C₁₋₆ haloalkyl, and C₁₋₃ alkoxy-C₁₋₃ alkoxy-;
R² is C₁₋₆ alkyl;
R³ is selected from H, halo, and C₁₋₆ alkoxy;
R^{c4} and R^{d4} are each independently selected from H, C₁₋₆ alkyl, C₁₋₆ haloalkyl, and C₃₋₁₀ cycloalkyl, wherein the C₁₋₆ alkyl and C₃₋₁₀ cycloalkyl are each optionally substituted with 1, 2, 3, or 4 substituents independently selected from halo, C₁₋₆ alkyl, C₁₋₆ haloalkyl, C₁₋₆ alkoxy, and phenylamino;
or R^{c4} and R^{d4}, together with the N atom to which they are attached, form a 4-10 membered heterocycloalkyl group, which is optionally substituted with 1, 2, 3, or 4 substituents independently selected from halo and C₁₋₆ alkoxy; and
R⁵ is selected from H, halo, and C₁₋₆ alkoxy.

2. The compound of claim 1, or a pharmaceutically acceptable salt thereof, wherein L¹ is —CH₂— or C(O).

3. The compound of claim 1, or a pharmaceutically acceptable salt thereof, wherein L¹ is —CH₂—.

4. The compound of claim 1, or a pharmaceutically acceptable salt thereof, wherein L¹ is C(O).

5. The compound of claim 1, or a pharmaceutically acceptable salt thereof, wherein R¹ is selected from phenyl, dihydrobenzofuranyl, dihydroisobenzofuranyl, and indazolyl, each of which is optionally substituted by 1 or 2 substituents independently selected from fluoro, methyl, isopropyl, and methoxyethoxy.

6. The compound of claim 1, or a pharmaceutically acceptable salt thereof, wherein R¹ is selected from fluorophenyl, (methoxyethoxy)phenyl, dimethyldihydrobenzofuranyl, dimethyldihydroisobenzofuranyl, and isopropylindazolyl.

7. The compound of claim 1, or a pharmaceutically acceptable salt thereof, wherein R² is C₁₋₃ alkyl.

8. The compound of claim 1, or a pharmaceutically acceptable salt thereof, wherein R² is methyl or trideuteromethyl.

9. The compound of claim 1, or a pharmaceutically acceptable salt thereof, wherein R³ is selected from H, halo, and C₁₋₃ alkoxy.

10. The compound of claim 1, or a pharmaceutically acceptable salt thereof, wherein R³ is selected from H, fluoro, and methoxy.

11. The compound of claim 1, or a pharmaceutically acceptable salt thereof, wherein R^{c4} and R^{d4} are each independently selected from H, C₁₋₆ alkyl, C₁₋₆ haloalkyl, and C₃₋₁₀ cycloalkyl, wherein the C₁₋₆ alkyl and C₃₋₁₀ cycloalkyl are each optionally substituted with 1, 2, 3, or 4 substituents independently selected from halo, C₁₋₆ alkyl, C₁₋₆ haloalkyl, C₁₋₆ alkoxy, and phenylamino.

12. The compound of claim 1, or a pharmaceutically acceptable salt thereof, wherein R^{c4} and R^{d4} are each independently selected from H, C₁₋₃ alkyl, C₁₋₃ haloalkyl, and C₃₋₇ cycloalkyl, wherein the C₁₋₃ alkyl and C₃₋₇ cycloalkyl are each optionally substituted with 1, 2, 3, or 4 substituents independently selected from halo, C₁₋₃ alkyl, C₁₋₃ haloalkyl, C₁₋₃ alkoxy, and phenylamino.

13. The compound of claim 1, or a pharmaceutically acceptable salt thereof, wherein R^{c4} and R^{d4} are each independently selected from H, methyl, trideuteromethyl, ethyl, propyl, isopropyl, trifluoroethyl, and cyclopropyl, wherein the methyl, ethyl, propyl, isopropyl, and cyclopropyl are each optionally substituted with 1, 2, 3, or 4 substituents independently selected from fluoro, trifluoromethyl, methoxy, and phenylamino.

14. The compound of claim 1, or a pharmaceutically acceptable salt thereof, wherein:
R^{c4} is selected from methyl, trideuteromethyl, ethyl, propyl, isopropyl, trifluoroethyl, and cyclopropyl, wherein the methyl, ethyl, propyl, isopropyl, and cyclopropyl are each optionally substituted with 1, 2, 3, or 4 substituents independently selected from fluoro, trifluoromethyl, methoxy, and phenylamino; and
R^{d4} is selected from H, methyl, and trideuteromethyl.

15. The compound of claim 1, or a pharmaceutically acceptable salt thereof, wherein:
R^{c4} is selected from methyl, trideuteromethyl, ethyl, isopropyl, trifluoroethyl, methoxyethyl, cyclopropyl, phenylaminopropyl, difluorocyclopropyl, and trifluoromethylcyclopropyl; and
R⁴⁴ is selected from H, methyl, and trideuteromethyl.

16. The compound of claim 1, or a pharmaceutically acceptable salt thereof, wherein R^{c4} and R^{d4}, together with the N atom to which they are attached, form a 4-10 membered heterocycloalkyl group, which is optionally substituted with 1, 2, 3, or 4 substituents independently selected from halo and C₁₋₆ alkoxy.

17. The compound of claim 1, or a pharmaceutically acceptable salt thereof, wherein R^{c4} and R^{d4}, together with the N atom to which they are attached, form a monocyclic 4-7 membered heterocycloalkyl group or a 8-10 membered bicyclic heterocycloalkyl group, wherein the monocyclic 4-7 membered heterocycloalkyl group or a 8-10 membered bicyclic heterocycloalkyl group are each optionally substituted with 1, 2, 3, or 4 substituents independently selected from halo and C₁₋₆ alkoxy.

18. The compound of claim 1, or a pharmaceutically acceptable salt thereof, wherein R^{c4} and R^{d4}, together with the N atom to which they are attached, form azetidinyl, piperidinyl, morpholinyl, or 3-oxa-8-azabicyclo[3.2.1]octanyl, wherein the azetidinyl, piperidinyl, morpholinyl, and 3-oxa-8-azabicyclo[3.2.1]octanyl are each optionally substituted with 1, 2, 3, or 4 substituents independently selected from fluoro and methoxy.

19. The compound of claim 1, or a pharmaceutically acceptable salt thereof, wherein R^{c4} and R^{d4}, together with the N atom to which they are attached, form methoxyazetidinyl, difluoropiperidinyl, morpholinyl, or 3-oxa-8-azabicyclo[3.2.1]octanyl.

20. The compound of claim 1, or a pharmaceutically acceptable salt thereof, wherein R⁵ is selected from H, halo, and C₁₋₃ alkoxy.

21. The compound of claim 1, or a pharmaceutically acceptable salt thereof, wherein R⁵ is selected from H, fluoro, and methoxy.

22. The compound of claim 1, or a pharmaceutically acceptable salt thereof, wherein:
L¹ is —CH₂— or C(O);
R¹ is selected from phenyl, dihydrobenzofuranyl, dihydroisobenzofuranyl, and indazolyl, each of which is optionally substituted by 1 or 2 substituents independently selected from fluoro, methyl, isopropyl, and methoxyethoxy;
R² is C₁₋₃ alkyl;
R^{c4} and R^{d4} are each independently selected from H, C₁₋₆ alkyl, C₁₋₆ haloalkyl, and C₃₋₁₀ cycloalkyl, wherein the C₁₋₆ alkyl and C₃₋₁₀ cycloalkyl are each optionally substituted with 1, 2, 3, or 4 substituents independently selected from halo, C₁₋₆ alkyl, C₁₋₆ haloalkyl, C₁₋₆ alkoxy, and phenylamino; or
R^{c4} and R^{d4}, together with the N atom to which they are attached, form a 4-10 membered heterocycloalkyl group, which is optionally substituted with 1, 2, 3, or 4 substituents independently selected from halo and C₁₋₆ alkoxy; and
R⁵ is selected from H, halo, and C₁₋₃ alkoxy.

23. The compound of claim 1, or a pharmaceutically acceptable salt thereof, wherein:
L¹ is —CH₂— or C(O);
R¹ is selected from phenyl, dihydrobenzofuranyl, dihydroisobenzofuranyl, and indazolyl, each of which is optionally substituted by 1 or 2 substituents independently selected from fluoro, methyl, isopropyl, and methoxyethoxy;
R² is C₁₋₃ alkyl;
R^{c4} and R^{d4} are each independently selected from H, C₁₋₃ alkyl, C₁₋₃ haloalkyl, and C₃₋₇ cycloalkyl, wherein the C₁₋₃ alkyl and C₃₋₇ cycloalkyl are each optionally substituted with 1, 2, 3, or 4 substituents independently selected from halo, C₁₋₃ alkyl, C₁₋₃ haloalkyl, C₁₋₃ alkoxy, and phenylamino; or
R^{c4} and R⁴⁴, together with the N atom to which they are attached, form a monocyclic 4-7 membered heterocycloalkyl group or a 8-10 membered bicyclic heterocycloalkyl group, wherein the monocyclic 4-7 membered heterocycloalkyl group or a 8-10 membered bicyclic heterocycloalkyl group are each optionally substituted with 1, 2, 3, or 4 substituents independently selected from halo and C₁₋₆ alkoxy; and
R⁵ is selected from H, halo, and C₁₋₃ alkoxy.

24. The compound of claim 1, or a pharmaceutically acceptable salt thereof, wherein:
L¹ is —CH₂— or C(O);
R¹ is selected from fluorophenyl, (methoxyethoxy)phenyl, dimethyldihydrobenzofuranyl, dimethyldihydroisobenzofuranyl, and isopropylindazolyl;
R² is methyl or trideuteromethyl;
R³ is selected from H, fluoro, and methoxy;
R^{c4} and R⁴⁴ are each independently selected from H, methyl, trideuteromethyl, ethyl, propyl, isopropyl, trifluoroethyl, and cyclopropyl, wherein the methyl, ethyl, propyl, isopropyl, and cyclopropyl are each optionally substituted with 1, 2, 3, or 4 substituents independently selected from fluoro, trifluoromethyl, methoxy, and phenylamino; or
R^{c4} and R⁴⁴, together with the N atom to which they are attached, form azetidinyl, piperidinyl, morpholinyl, or 3-oxa-8-azabicyclo[3.2.1]octanyl, wherein the azetidinyl, piperidinyl, morpholinyl, and 3-oxa-8-azabicyclo[3.2.1]octanyl are each optionally substituted with 1, 2, 3, or 4 substituents independently selected from fluoro and methoxy; and
R⁵ is selected from H, fluoro, and methoxy.

25. The compound of claim 1, wherein the compound of Formula I is a compound of Formula Ia: 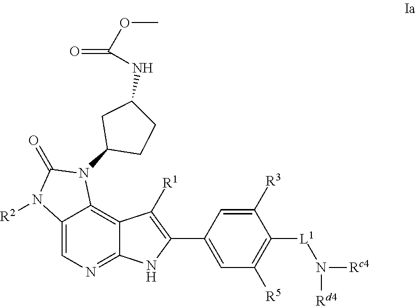
or a pharmaceutically acceptable salt thereof.

26. The compound of claim 1, wherein the compound of Formula I is a compound of Formula II: 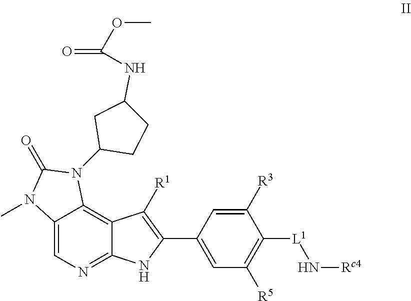
or a pharmaceutically acceptable salt thereof.

27. The compound of claim 1, wherein the compound of Formula I is a compound of Formula III: 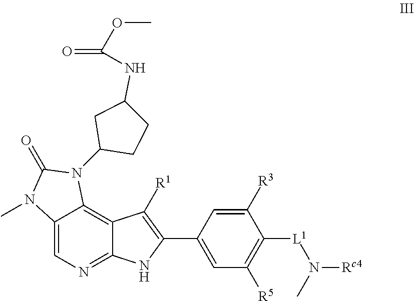
or a pharmaceutically acceptable salt thereof.

28. The compound of claim 1, wherein the compound of Formula I is a compound of Formula IV: 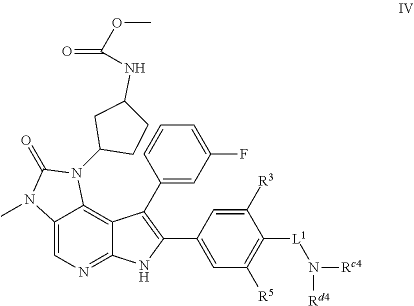
or a pharmaceutically acceptable salt thereof.

29. The compound of claim 1, wherein the compound of Formula I is a compound of Formula V: 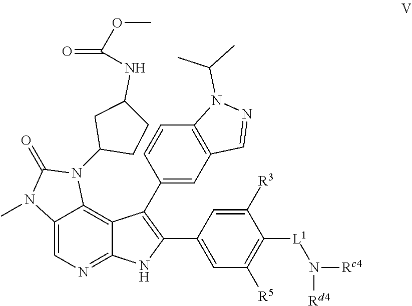
or a pharmaceutically acceptable salt thereof.

30. The compound of claim 1, wherein the compound of Formula I is a compound of Formula VI: 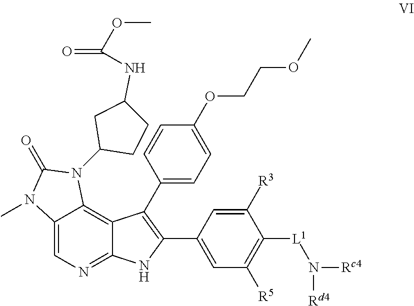
or a pharmaceutically acceptable salt thereof.

31. The compound of claim 1, wherein the compound of Formula I is a compound of Formula VII: 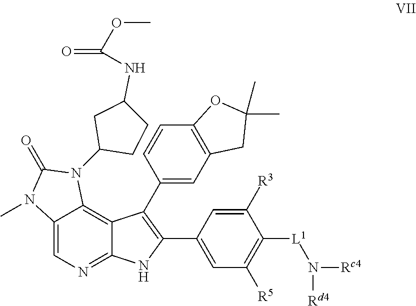
or a pharmaceutically acceptable salt thereof.

32. The compound of claim 1, wherein the compound of Formula I is a compound of Formula VIII: 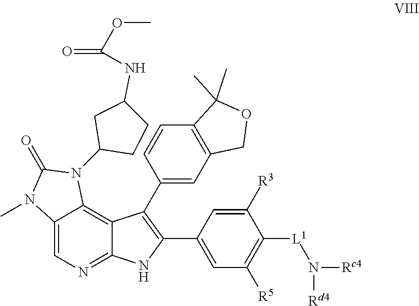
or a pharmaceutically acceptable salt thereof.

33. The compound of claim 1, which is selected from:
methyl ((1R,3R)-3-(7-(3-fluoro-4-(methylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(isopropylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3,5-difluoro-4-(methylcarbamoyl)phenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(cyclopropylcarbamoyl)-3,5-difluorophenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(3-oxa-8-azabicyclo[3.2.1]octane-8-carbonyl)-3-methoxyphenyl)-8-(3-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(ethylcarbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((3-(phenylamino)propyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(cyclopropylcarbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-((2,2-difluorocyclopropyl)carbamoyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2-(trifluoromethyl)cyclopropyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2,2,2-trifluoroethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(methylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((1-(trifluoromethyl)cyclopropyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2-methoxyethyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-((2-methoxyethyl)(methyl)carbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(morpholine-4-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(3-fluoro-4-(3-methoxyazetidine-1-carbonyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(ethylcarbamoyl)-3-methoxyphenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(8-(1-isopropyl-1H-indazol-5-yl)-7-(4-((2-methoxyethyl)carbamoyl)phenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(8-(1-isopropyl-1H-indazol-5-yl)-3-methyl-7-(4-(morpholine-4-carbonyl)phenyl)-2-oxo-3,6-dihydroimidazo[4,5-d]pyrrolo[2,3-b]pyridin-1(2H)-yl)cyclopentyl)carbamate;
methyl ((1R,3R)-3-(8-(1-isopropyl-1H-indazol-5-yl)-3-methyl-7-(4-(methylcarbamoyl)phenyl)-2-oxo-3,6-dihydroimidazo[4,5-d]pyrrolo[2,3-b]pyridin-1(2H)-yl)cyclopentyl)carbamate;
methyl ((1R,3R)-3-(7-(4-(dimethylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(ethylcarbamoyl)phenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-(4,4-difluoropiperidine-1-carbonyl)-3-fluorophenyl)-8-(1-isopropyl-1H-indazol-5-yl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-((bis(methyl-d3)amino)methyl)phenyl)-8-(2,2-dimethyl-2,3-dihydrobenzofuran-5-yl)-3-(methyl-d3)-2-oxo-3,6-dihydroimidazo[4,5-d]pyrrolo[2,3-b]pyridin-1(2H)-yl)cyclopentyl)carbamate;
methyl ((1R,3R)-3-(7-(4-((dimethylamino)methyl)phenyl)-8-(1-isopropyl-1H-indazol-5-yl)-3-(methyl-d3)-2-oxo-3,6-dihydroimidazo[4,5-d]pyrrolo[2,3-b]pyridin-1(2H)-yl)cyclopentyl)carbamate;
methyl ((1R,3R)-3-(7-(4-((bis(methyl-d3)amino)methyl)phenyl)-8-(4-(2-methoxyethoxy)phenyl)-3-(methyl-d3)-2-oxo-3,6-dihydroimidazo[4,5-d]pyrrolo[2,3-b]pyridin-1(2H)-yl)cyclopentyl)carbamate;
methyl ((1R,3R)-3-(7-(4-((dimethylamino)methyl)phenyl)-8-(4-fluorophenyl)-3-methyl-2-oxo-3,6-dihydroimidazo[4,5-d]pyrrolo[2,3-b]pyridin-1(2H)-yl)cyclopentyl)carbamate;
methyl ((1R,3R)-3-(7-(4-((bis(methyl-d3)amino)methyl)phenyl)-8-(1,1-dimethyl-1,3-dihydroisobenzofuran-5-yl)-3-(methyl-d3)-2-oxo-3,6-dihydroimidazo[4,5-d]pyrrolo[2,3-b]pyridin-1(2H)-yl)cyclopentyl)carbamate; and
methyl ((1R,3R)-3-(8-(1,1-dimethyl-1,3-dihydroisobenzofuran-5-yl)-7-(4-(dimethylcarbamoyl)phenyl)-3-(methyl-d3)-2-oxo-3,6-dihydroimidazo[4,5-d]pyrrolo[2,3-b]pyridin-1(2H)-yl)cyclopentyl)carbamate
or a pharmaceutically acceptable salt thereof.

34. The compound of claim 1, or a pharmaceutically acceptable salt thereof, wherein the compound is deuterated.

35. A pharmaceutical composition, comprising a compound of claim 1, or a pharmaceutically acceptable salt thereof, and a pharmaceutically acceptable carrier.

36. A method of inhibiting an activity of the V617F variant of JAK2 kinase, comprising contacting the kinase with a compound of claim 1, or a pharmaceutically acceptable salt thereof.

37. A method of treating cancer in a patient in need thereof, the method comprising administering to the patient a therapeutically effective amount of a compound of claim 1, or a pharmaceutically acceptable salt thereof.

38. The method of claim 37, wherein the cancer is selected from bladder cancer, breast cancer, cervical cancer, colorectal cancer, cancer of the small intestine, colon cancer, rectal cancer, cancer of the anus, endometrial cancer, gastric cancer, head and neck cancer, kidney cancer, liver cancer, lung cancer, ovarian cancer, prostate cancer, testicular cancer, uterine cancer, vulvar cancer, esophageal cancer, gall bladder cancer, pancreatic cancer, stomach cancer, thyroid cancer, parathyroid cancer, neuroendocrine cancer, skin cancer, and brain cancer.

39. The method of claim 37, wherein the cancer is a hematological cancer.

40. The method of claim 37, wherein the cancer is selected from leukemia, lymphoma, multiple myeloma, chronic lymphocytic lymphoma, adult T cell leukemia, acute myeloid leukemia, B-cell lymphoma, cutaneous T-cell lymphoma, acute myelogenous leukemia, Hodgkin's or non-Hodgkin's lymphoma, a myeloproliferative neoplasm), myelodysplastic syndrome, chronic eosinophilic leukemia, Waldenstrom's Macroglubulinemia, hairy cell lymphoma, chronic myelogenic lymphoma, acute lymphoblastic lymphoma, AIDS-related lymphoma, and Burkitt's lymphoma.

41. A method of treating a myeloproliferative disorder in a patient in need thereof, the method comprising administering to the patient a therapeutically effective amount of a compound of claim 1, or a pharmaceutically acceptable salt thereof.

42. The method of claim 41, wherein the myeloproliferative disorder is selected from polycythemia vera, essential thrombocythemia, myelofibrosis with myeloid metaplasia, primary myelofibrosis, post-essential thrombocythemia myelofibrosis, post polycythemia vera myelofibrosis, chronic myelogenous leukemia, chronic myelomonocytic leukemia, hypereosinophilic syndrome, and systemic mast cell disease.

43. A method of treating myelodysplastic syndrome in a patient in need thereof, the method comprising administering to the patient a therapeutically effective amount of a compound of claim 1, or a pharmaceutically acceptable salt thereof.
